(19) United States
(12) Patent Application Publication
(10) Pub. No.: US 2009/0264394 A1
(43) Pub. Date: Oct. 22, 2009

(54) NITROOXY DERIVATIVES OF GLUCOCORTICOIDS

(75) Inventors: Francesca Benedini, San Donato Milanese (MI) (IT); Ennio Ongini, Segrate (MI) (IT); Antonio Guglietta, Barcelona (ES); Daniel Palop, Barcelona (ES); Marta Princep, Barcelona (ES)

Correspondence Address: ARENT FOX LLP, 1050 CONNECTICUT AVENUE, N.W., SUITE 400, WASHINGTON, DC, 20036, US

(73) Assignees: Nicox S.A., Sophia Antipolis-Valbonne (FR); Ferrer Internacional S.A., Barcelona (ES)

(21) Appl. No.: 12/492,404

(22) Filed: Jun. 26, 2009

Related U.S. Application Data
(63) Continuation of application No. 12/065,561, filed on Mar. 3, 2008.

(30) Foreign Application Priority Data
Sep. 2, 2005 (EP) 05019155.0

(51) Int. Cl. A61K 31/58; C07J 71/00; A61P 17/00

(52) U.S. Cl. 514/174; 540/70

(57) ABSTRACT

The invention relates to new steroids nitrooxyderivatives, to topical pharmaceutical formulations thereof, and their use for treating skin or mucosal membrane diseases or disorders. These new steroids nitrooxyderivatives have an improved pharmacological activity and enhanced local tolerability.

### TECHNICAL FIELD

The present invention relates to new steroids nitrooxyderivatives, to topical pharmaceutical formulations thereof, and their use for treating skin or mucosal membrane diseases or disorders.

### BACKGROUND ART

Most of the skin or mucosal membrane diseases or disorders are the result of inflammation caused by inflammatory agents, such as, but not limited to, bacterial, fungal, viral, parasitic, autoimmune, allergic, hormonal and/or malignant inflammatory agents. The most common skin diseases or disorders include, but is not limited to, corticosteroid-responsive dermatosis, atopic dermatitis, inflammation, eczema, erythema, papulation, scaling, erosion, oozing, crusting, pruritis, psoriasis, epidermalysis bullosa, erythema, hidradenitis suppurative, warts, diaper rash, jock itch, ruber lichen planus.

Dermatitis and eczema result from inflammatory processes that involve the upper dermis and epidermis of the skin. When eczema develops, the keratinocytes in the epidermis distend from one another and fluid is accumulated there amongst in a process known as spongiosis.

In chronic forms of eczema or dermatitis the main change include thickening of the epidermis, which leads to itching, roughening and scaling of the skin surface. The loss of water from the skin leads to inflammation of the horny layer, which later results in cracked and sore skin. Dermatitis is further classified into contact dermatitis (allergic or non allergic), atopic dermatitis and seborrheic dermatitis. Non-allergic contact dermatitis occurs in response to skin irritants, such as acids, alkalis, oils, detergents and solvents.

Allergic contact dermatitis occurs as a result of sensitization to repeated exposure to an antigen. Allergic contact dermatitis appears in skin areas that were in direct contact with the antigen.

Atopic dermatitis, which affects mainly infants, is characterized by sensitization of the skin to a wide range of common antigens.

Seborrheic dermatitis affects the scalp and other hairy areas, the face, and flexural areas and results from yeast or bacteria induced inflammation. Most people suffer from dandruff that is a mild form of seborrheic dermatitis. Psoriasis is a dominant autosomal inherited inflammatory disease characterized by enhanced proliferation of keratinocytes which proliferation leads to formation of scaly plaques on, for example, the knees, elbows, buttocks, and which are aesthetically unpleasant and cause discomfort to the affected subject.

Skin diseases or disorders are usually treated by creams, gels or ointments containing steroidal agents and/or antibacterial agents and/or antifungal agents.

Topical corticosteroids are a powerful tool for treating skin disease.

In clinical practice, for example the use of super potent topical steroids is typically limited to only two weeks because of their use may be associated with adverse side effects such as skin atrophy, burning, itching, irritation, dryness, folliculitis, hypertrichosis, acne, hype pigmentation, perioral dermatitis, allergic contact dermatitis, maceration of the skin, and secondary infection.

Although topical administration of corticosteroids minimizes the side-effects as compared to systemic administration, the active compounds are still absorbed into the circulation where they are systemically active.

Systemic absorption of topical corticosteroids can result in reversible hypothalamic-pituitary-adrenal (HPA) axis suppression, Cushing's syndrome-like symptoms, hyperglycemia, effects on bone growth in children and on bone density in the elderly, ocular complications (cataract formation and glaucoma) and skin atrophy.

Furthermore, tachyphylaxis may result from the use of the topical steroid.

Whilst the modern glucocorticoids are very much safer than those originally introduced, it remains an object of research to produce new molecules and formulations having an improved clinical efficacy, and reduced side effects.

A variety of protocols have been developed to try to increase the efficiency and/or effectiveness of a topical agent, although thus far such protocols have met with limited success. For example, dermatological agents have been provided in a variety of topical formulations such as creams, lotions, gels and the like in attempts to increase the delivery efficiency. However, while enabling direct, localized application of the dermatological agent to a skin surface, these topical formulations have not provided a complete solution as typically only partial improvement results even with an optimal formulation, e.g., oftentimes recalcitrant skin lesions remain, and/or treatment times have not been appreciably shortened.

U.S. Pat. No. 4,335,121 discloses 6.alpha., 9.alpha.-Difluoro-17.alpha.-(1-oxopropoxy)-11.beta.-hydroxy-16.alpha.-methyl-3-oxo-androsta-1,4-diene-17-0-carbothioic acid S-fluoromethyl ester (known by the generic name of fluticasone propionate) and derivatives thereof, these compounds have good anti-inflammatory activity, particularly on topical applications.

EP 0929565 discloses nitroxyesters of corticosteroids that among systemic uses can be used for the treatment of dermatological disorders; in particular the patent discloses nitroxyesters of corticosteroids in which the nitroxy group is covalently linked through an alkyl chain to the glucocorticoid moiety. The document reports that these nitroderivatives of steroids, after systemic administration, displayed enhanced efficacy and better systemic tolerability, such as better gastric tolerability, reduced cardiovascular side effects, compared with their parent compounds.

WO03/064443 discloses nitrooxyderivatives of corticosteroids in which the nitrooxy group is covalently linked through an aromatic or a heteroarylic ring containing linker to the glucocorticoid moiety. The document reports that these nitrooxyderivatives of steroids, after systemic administration, displayed an improved pharmacological activity and lower side effect compared to their parent compounds.

WO00/61604 discloses nitrooxyderivatives of corticosteroids in which the nitrooxy group is covalently linked through an “antioxidant moiety” to the glucocorticoid moiety, such “antioxidant moieties” are compounds capable to prevent the production of free radicals and are selected on the basis of tests described in the patent application. The document reports that these compounds can be used for the treatment of pathologies associated with an oxidative stress condition in which the corresponding parent compounds show lower activity or higher toxicity.

The above-mentioned documents do not disclose the activity of the nitrooxyderivatives of corticosteroids after topical administration and in particular do not report any information regarding the local tolerability of the compounds.

WO 97/34871 discloses nitrosated or nitrosilate steroids and their use for the treatment of respiratory disorder, in particular describe the activity in a pulmonary model of allergic asthma and lung inflammation of 9-fluoro-11β-hydroxy-16α,17α-[(1-methylethylidene)bis(oxy)]pregna-1,4-diene-3,20-dione-21(4-nitrooxy)-butanoate. The patent application does not mention the use of the compounds in treatment of skin disorders.

Hyun E. et al, British Journal of Pharmacology (2004) 143, 618-625, relates to a study of the activity of hydrocortisone 21-[4′-(nitrooxymethyl)benzoate] in a model of irritant acute dermatitis, in this study oedema formation and recruitment of leukocytes were evaluated and the results demonstrate that the compound has a higher anti-inflammatory activity than the parent compound hydrocortisone. The document does not report any information regarding the effect of the compound on the skin after a long-lasting treatment. Moreover the experimental model described by Hyun E. et al is not predictive for other dermatological disorders.

### DISCLOSURE OF THE INVENTION

The present invention solves the above-mentioned problems by providing new nitrooxyderivatives of corticosteroids having an improved pharmacologically profile, better pharmacokinetic and pharmacodynamic properties and fewer adverse side effects, in particular the compounds of the invention show an improved local tolerability, such as reduction of skin blanching and skin atrophy, a fast onset of action and an increased efficacy than the existing topical corticosteroids. In particular the nitrooxyderivatives of corticosteroids of the present invention are more effective than the parent drugs in reducing local inflammation mediated vasodilatation resulting in a reduction of oedema and of the infiltration of inflammatory mediators.

An object of the present invention is compounds of general formula (I)

R-Z-X—ONO₂ (I)

wherein R is the corticosteroid residue of formula (II):

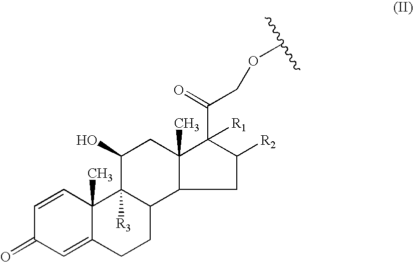

wherein
R₁ is —OC(O)OₘRᵢ wherein m is 0 or 1, Rᵢ is a branched or straight C₁-C₁₀ alkyl, preferably R₁ is a branched or straight C₁-C₆ alkyl, preferred R₁ groups are: methyl, ethyl, n-propyl, n-butyl;
R₂ is an hydrogen atom or —CH₃;
or R₁ and R₂ when taken together are the group of formula (III)

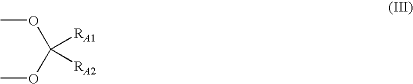

wherein R_{A1} and R_{A2} are independently selected from H, a C₁-C₁₀ linear or branched alkyl chain, preferably (C₁-C₅) alkyl; more preferably R_{A1} and R_{A2} are —CH₃ and the group of formula (III) is a isopropylidenedioxy;
R₁ and R₂ can be linked to the carbon atoms in 16 and 17 of the steroidal structure in position α or β;
R₃ is a hydrogen atom or a fluorine atom;
preferably in formula (II) R₁, R₂ and R₃ have the following meanings:

R₁ and R₂ both in position α are taken together and form the group of formula (III) wherein R_{A1} and R_{A2} are —CH₃, R₃ is a fluorine atom; or

R₁ is —OC(O)OₘRᵢ in position α wherein m is 1 and Rᵢ is ethyl and R₂ and R₃ are hydrogen atoms; or

R₁ is —OC(O)OₘRᵢ in position α wherein m is 0, Rᵢ is n-butyl, R₂ is —CH₃ in position A, R₃ is a fluorine atom;

Z is a group capable of binding X selected from the group consisting of:

### —C(O)—, —C(O)O— or

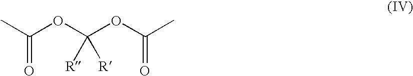

wherein R′ and R″ are independently selected from H or straight or branched C₁-C₄ alkyl; preferably Z is —C(O)— or —C(O)O—;
X is a bivalent radical having the following meanings:
a) straight or branched C₁-C₂₀ alkylene, preferably a straight or branched C₁-C₁₀ alkylene, being optionally substituted with one or more of the substituents selected from the group consisting of: halogen atoms, hydroxy, —ONO₂ or T, wherein T is —OC(O)(C₁-C₁₀ alkyl)-ONO₂ or —O(C₁-C₁₀ alkyl)-ONO₂; preferably X is a straight C₁-C₁₀ alkylene;
b) a C₅-C₇ cycloalkylene group optionally substituted with linear or branched C₁-C₁₀ alkyl group, preferably CH₃;
c)

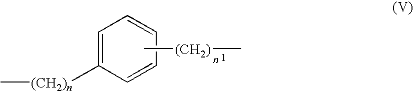

d)

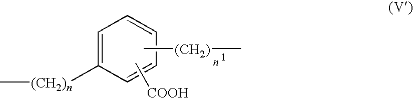

wherein n is an integer from 0 to 20, preferably n is an integer from 0 to 5; more preferably n is 0 or 1;
n¹ is an integer from 1 to 20, preferably n¹ is an integer from 1 to 5; more preferably n¹ is 1;
e)

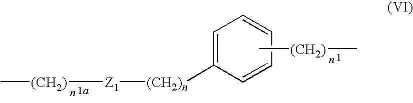

wherein n^{1a} is an integer from 1 to 20, preferably n^{1a} is an integer from 1 to 10;
Z₁ is —C(O)O— or —OC(O)—; preferably Z₁ is —C(O)O—;
n is as above defined;
n¹ is as above defined; preferably in formula (VI) n^{1a} is an integer from 1 to 10;
Z₁ is —C(O)O—, n is 0 or 1 and n¹ is 1;
with the proviso that when X is selected from the bivalent radicals mentioned under c)-e), the —ONO₂ group of formula (I) is linked to the —(CH₂)ₙ¹— group;
f)

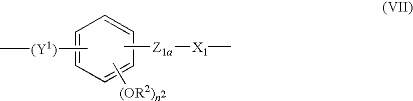

wherein:
Y¹ is —CH₂—CH₂—(CH₂)ₙ^{2a}—, or —CH═CH— (CH₂)ₙ^{2a}— wherein and n^{2a} is an integer from 0 to 10; preferably n^{2a} is 0 or is an integer from 1 to 6;

### Z₁ₐ is —OC(O)— or —C(O)O—;

n² is 0 or 1; preferably n² is 1;
R² is H or CH₃; preferably R² is CH₃;
X₁ is —(CH)ₙ^{1a}— wherein n^{1a} is as above defined, or the bivalent radical of formula (V) wherein n and n¹ are as above defined;
preferably in formula (VII) Y¹ is —CH═CH—(CH₂)ₙ^{2a}— wherein n^{2a} is 0, Z₁ₐ is —OC(O)—, n² is 1, R² is CH₃, X₁ is —(CH)ₙ^{1a}—, wherein n^{1a} is an integer from 1 to 10;
with the proviso that in formula (VII) the —ONO₂ group of formula (I) is linked to the X₁ group;
g)

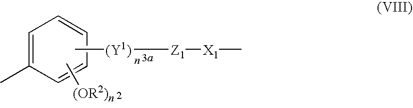

wherein:
Y¹ is —CH₂—CH₂—(CH₂)ₙ^{2a}—, or —CH═CH—(CH₂)ₙ^{2a}— wherein and n^{2a} is an integer from 0 to 10; preferably n^{2a} is 0 or n^{2a} is an integer from 1 to 6;
n^{3a} is 0 or 1;

### Z₁ is —C(O)O— or —OC(O)—;

n² is 0 or 1; preferably n² is 1;
R² is H or CH₃; preferably R² is CH₃;
X₁ is —(CH)ₙ^{1a}— wherein n^{1a} is as above defined, or the bivalent radical of formula (V) wherein n and n¹ are as above defined;
preferably in formula (VIII) n^{3a} is 1, Y¹ is —CH═CH—(CH₂)ₙ^{2a}— wherein n² is 0, Z₁ is —C(O)O—, n² is 1, R² is CH₃, X₁ is —(CH)ₙ^{1a}— wherein n^{1a} is an integer from 1 to 10; or in formula (VIII) n^{3a} is 0, Z₁ is —OC(O)— or —C(O)O—, n² is 1, R² is CH₃ and X₁ is —(CH)ₙ^{1a}— wherein n^{1a} is an integer from 1 to 10;
with the proviso that in formula (VIII) the —ONO₂ group of formula (I) is linked to the X₁ group;
h)

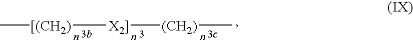

wherein
X₂ is —O— or —S—, preferably X₂ is —O—;
n³ is an integer from 1 to 6, preferably from 1 to 4, and
n^{3b} is an integer from 1 to 10, preferably from 1 to 6, more preferably n^{3b} is 1 or 2;
n^{3c} is an integer from 1 to 10, preferably from 1 to 6, more preferably n^{3c} is 2;
i)

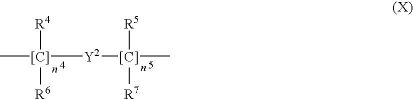

wherein:
n⁴ is an integer from 0 to 10;
n⁵ is an integer from 1 to 10;
R⁴, R⁵, R⁶, R⁷ are the same or different, and are H or straight or branched C₁-C₄ alkyl, preferably R⁴, R⁵, R⁶, R⁷ are H;
wherein the —ONO₂ group of formula (I) is linked to

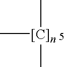

wherein n⁵ is as defined above;
Y² is an heterocyclic saturated, unsaturated or aromatic 5 or 6 members ring, containing one or more heteroatoms selected from nitrogen, oxygen, sulfur,
and is selected from

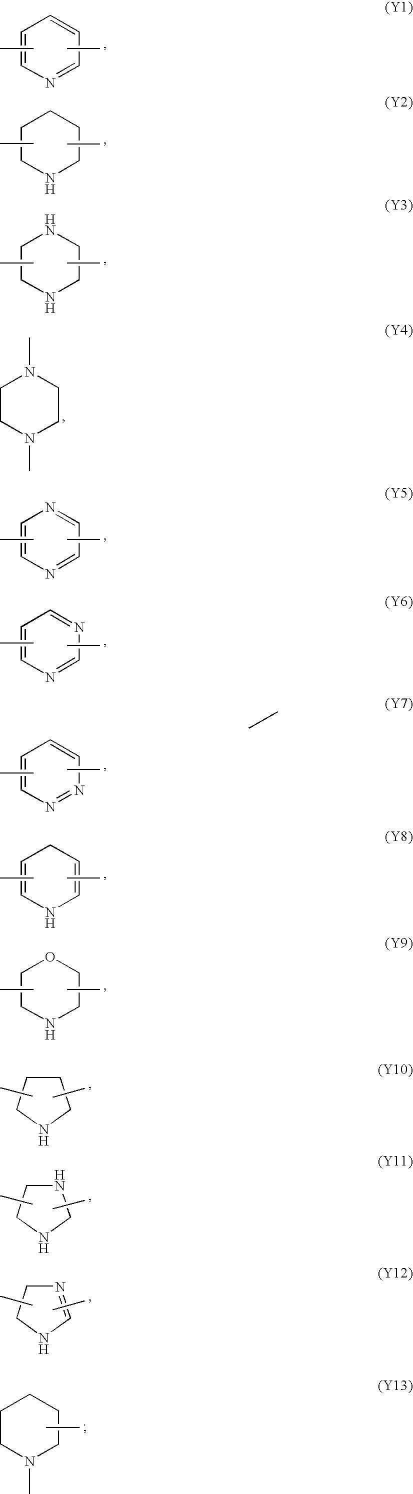

with the proviso that when in formula (I) Z is —C(O)— and in formula (II) R₁ and R₂ both in position α are taken together and forms the group of formula (III) wherein R_{A1} and R_{A2} are —CH₃, R₃ is a fluorine atom, then X has not the following meaning:
a) straight or branched C₁-C₂₀ alkylene, preferably a straight or branched C₁-C₁₀ alkylene, being optionally substituted with one or more of the substituents selected from the group consisting of: halogen atoms, hydroxy, —ONO₂ or T, wherein T is —OC(O)(C₁-C₁₀ alkyl)-ONO₂ or —O(C₁-C₁₀ alkyl)-ONO₂; preferably X is a straight C₁-C₁₀ alkylene; preferred bivalent radicals X are:
a) straight C₁-C₁₀ alkylene;
c)

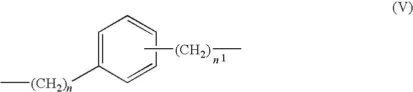

wherein n is 0 or 1 and n′ is 1;
e)

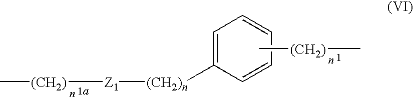

wherein n^{1a} is an integer from 1 to 10, Z₁ is —C(O)O— or —OC(O)—, n is 0 or 1 and n¹ is 1;
with the proviso that when X is selected from the bivalent radicals mentioned under c)-e), the —ONO₂ group of formula (I) is linked to the —(CH₂)ₙ¹— group;
f)

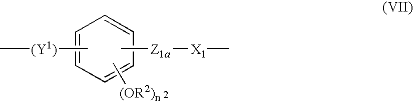

wherein:
Y¹ is —CH═CH—(CH₂)ₙ^{2a}— wherein n^{2a} is 0, Z₁ₐ is —OC(O)—, n² is 1, R² is CH₃, X is — (CH)ₙ^{1a}—, wherein n^{1a} is an integer from 1 to 10;
with the proviso that in formula (VII) the —ONO₂ group of formula (I) is linked to the X₁ group;
g)

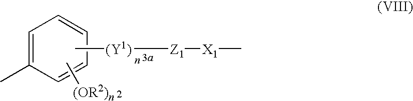

wherein:
n^{3a} is 1, Y¹ is —CH═CH—(CH₂)ₙ^{2a}— wherein n^{2a} is 0, Z₁ is —C(O)O—, n² is 1, R² is CH₃, X₁ is —(CH)ₙ^{1a}— wherein n^{1a} is an integer from 1 to 10;
or in formula (VIII) n^{3a} is 0, Z₁ is —OC(O)— or —C(O)O—, n² is 1, R² is CH₃ and X₁ is —(CH)ₙ^{1a}— wherein n^{1a} is an integer from 1 to 10;
with the proviso that in formula (VIII) the —ONO₂ group of formula (I) is linked to the X₁ group;
h)

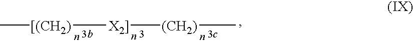

wherein

### X₂ is —O—;

n³ is an integer from 1 to 4;
n^{3b} is 1 or 2;
n^{3c} is 2;

One preferred embodiment of the present invention is compounds of formula (I)

R-Z-X—ONO₂ (I)

wherein R is the corticosteroid residue of formula (II) above reported wherein:

R₁ and R₂ both in position α are taken together and forms the group of formula (III) wherein R_{A1} and R_{A2} are —CH₃, R₃ is a fluorine atom; or

R₁ is —OC(O)OₘRᵢ in position α wherein m is 1 and Rᵢ is ethyl, R₂ and R₃ are hydrogen atoms; or

R₁ is —OC(O)OₘRᵢ in position α wherein m is 0, Rᵢ is n-butyl, R₂ is —CH₃ in position β, R₃ is a fluorine atom;

### Z is —C(O)— or —C(O)O—;

X has the following meanings:
a) straight C₁-C₁₀ alkylene;
c)

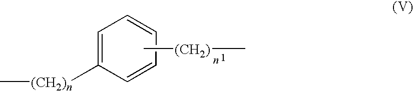

wherein n is 0 or 1 and n¹ is 1;
e)

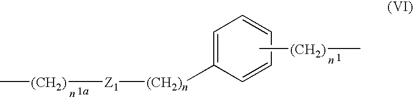

wherein n^{1a} is an integer from 1 to 10, Z₁ is —C(O)O— or —OC(O)—, n is 0 or 1 and n¹ is 1;
with the proviso that when X is selected from the bivalent radicals mentioned under c)-e), the —ONO₂ group of formula (I) is linked to the —(CH₂)ₙ¹— group;
f)

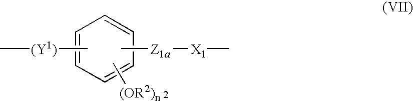

wherein:
Y¹ is —CH═CH—(CH₂)ₙ^{2a}— wherein n^{2a} is 0, Z₁ₐ is —OC(O)—, n² is 1, R² is CH₃, X₁ is —(CH)ₙ^{1a}—, wherein n^{1a} is an integer from 1 to 10;
with the proviso that in formula (VII) the —ONO₂ group of formula (I) is linked to the X₁ group;
g)

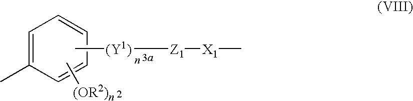

wherein:
n^{3a} is 1, Y¹ is —CH═CH—(CH₂)ₙ^{2a}— wherein n^{2a} is 0, Z₁ is —C(O)O—, n² is 1, R² is CH₃, X₁ is —(CH)ₙ^{1a}— wherein n^{1a} is an integer from 1 to 10;
or in formula (VIII) n^{3a} is 0, Z₁ is —OC(O)— or —C(O)O—, n² is 1, R² is CH₃ and X₁ is —(CH)ₙ^{1a}— wherein n^{1a} is an integer from 1 to 10;
with the proviso that in formula (VIII) the —ONO₂ group of formula (I) is linked to the X₁ group;
h)

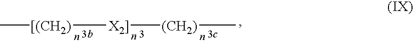

wherein

### X₂ is —O—;

n³ is an integer from 1 to 6, preferably from 1 to 4;
n^{3b} is 1 or 2;
n^{3c} is 2;
with the proviso that when in formula (I) Z is —C(O)— and in formula (II) R₁ and R₂ both in position (x are taken together and forms the group of formula (III) wherein R_{A1} and R_{A2} are —CH₃, R₃ is a fluorine atom, then X can not be straight C₁-C₁₀ alkylene;

The most preferred compounds of formula (I) of the present invention are:

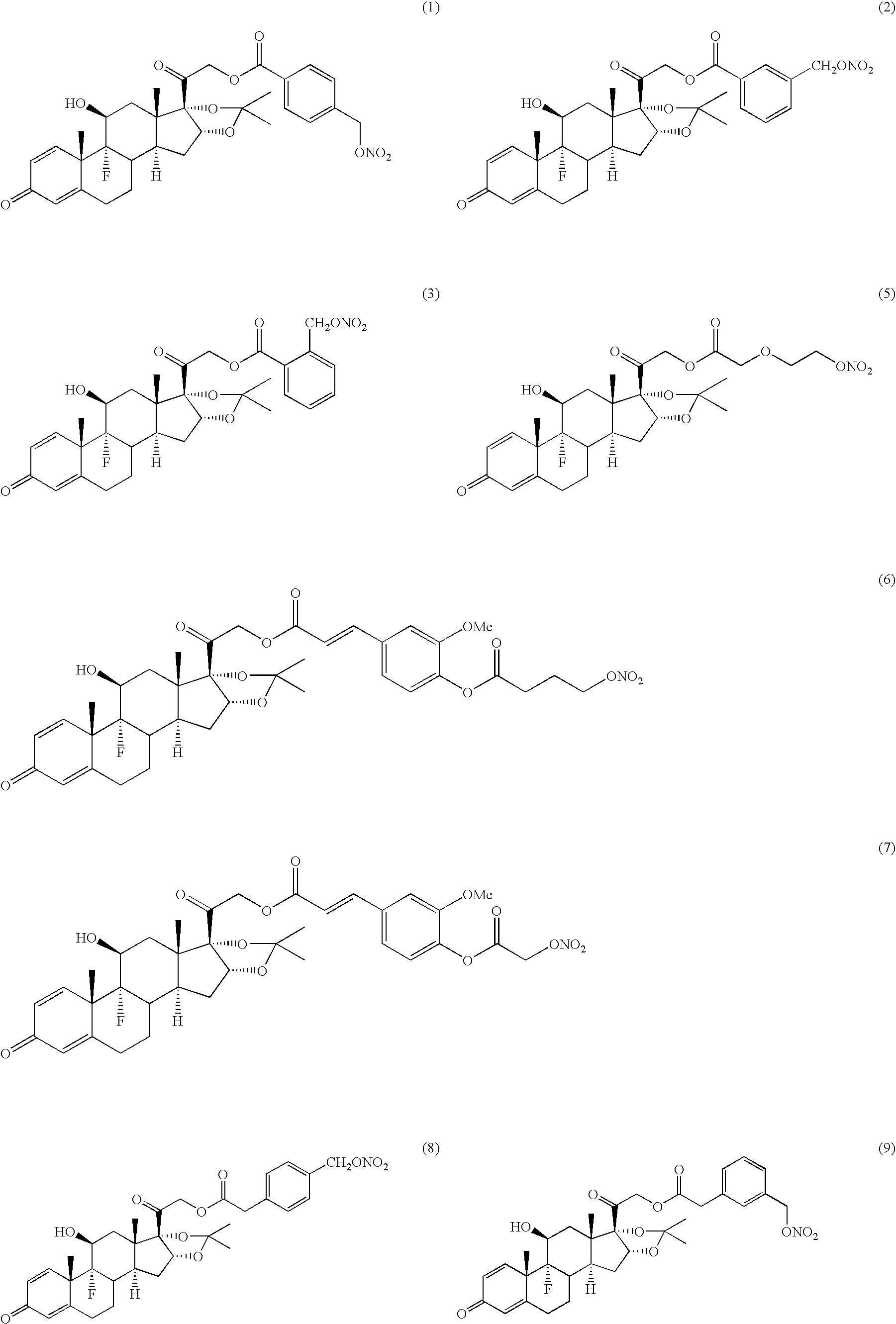

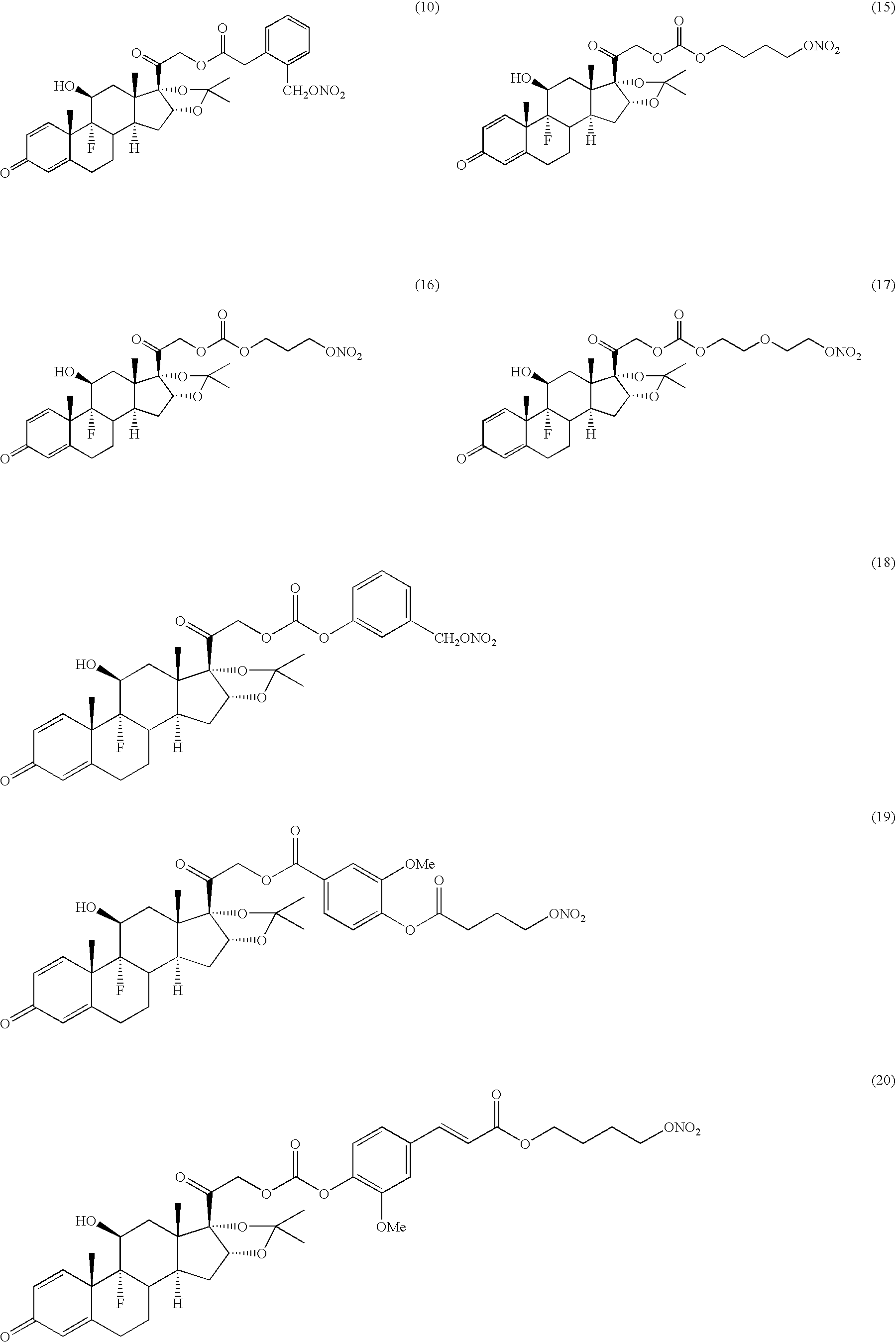

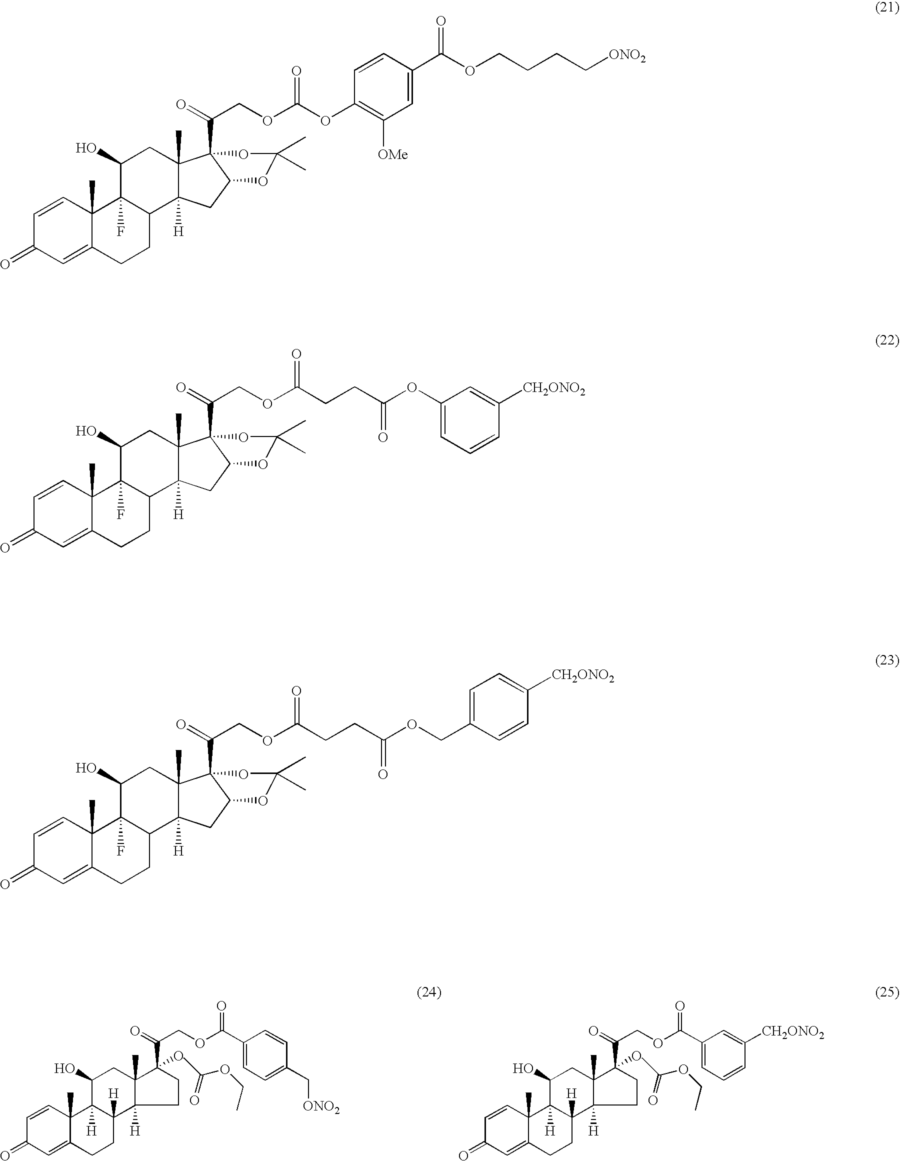

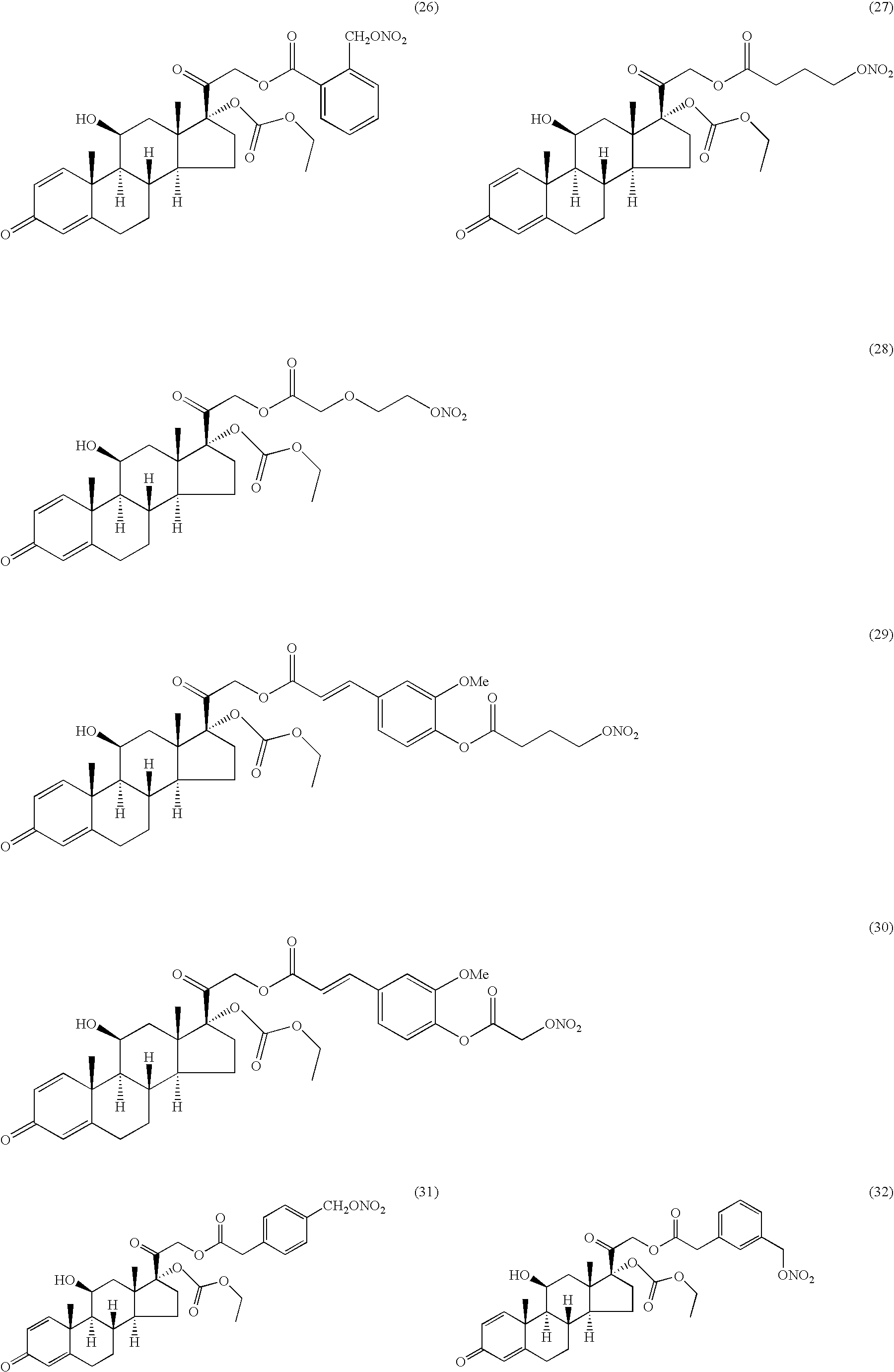

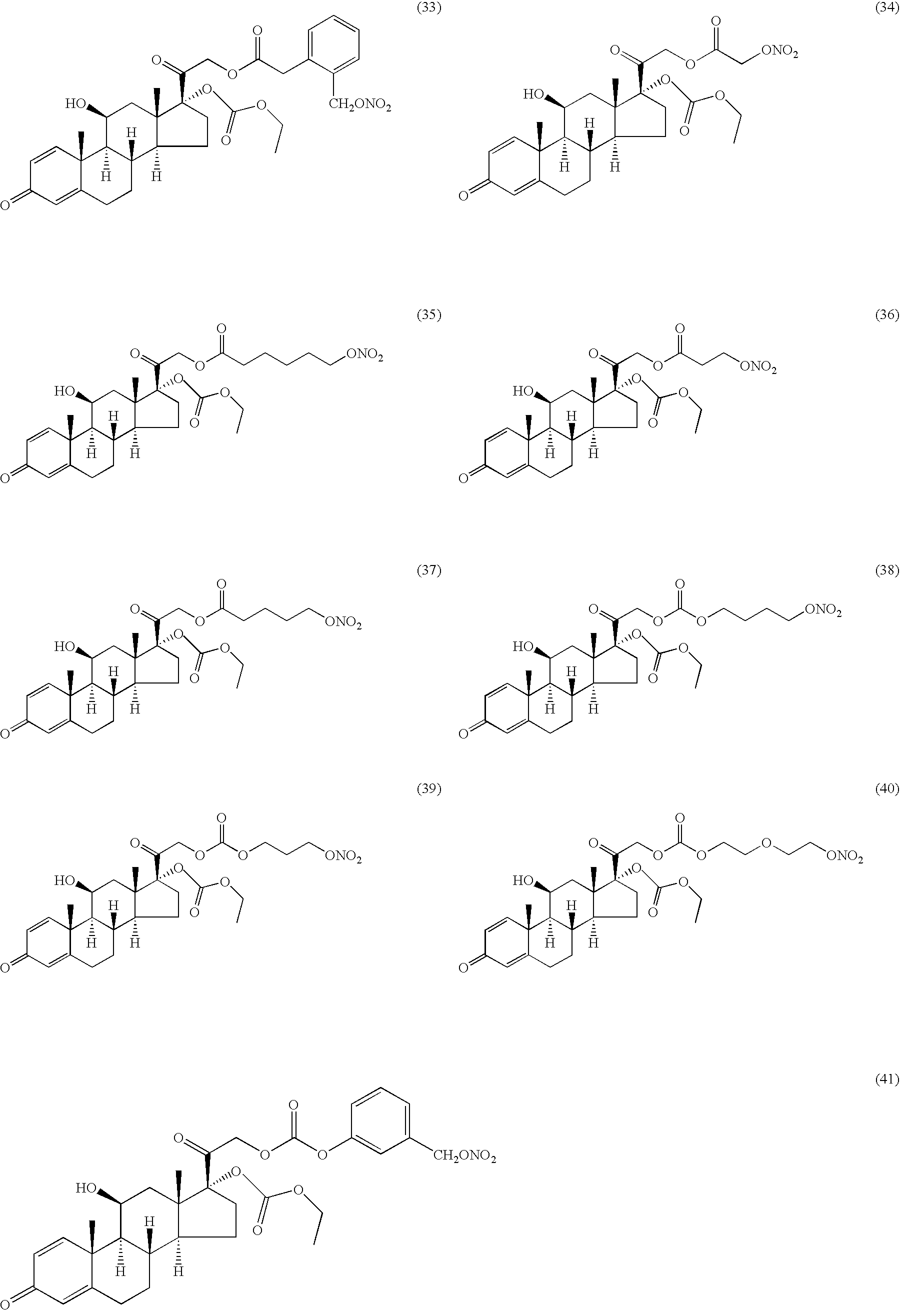

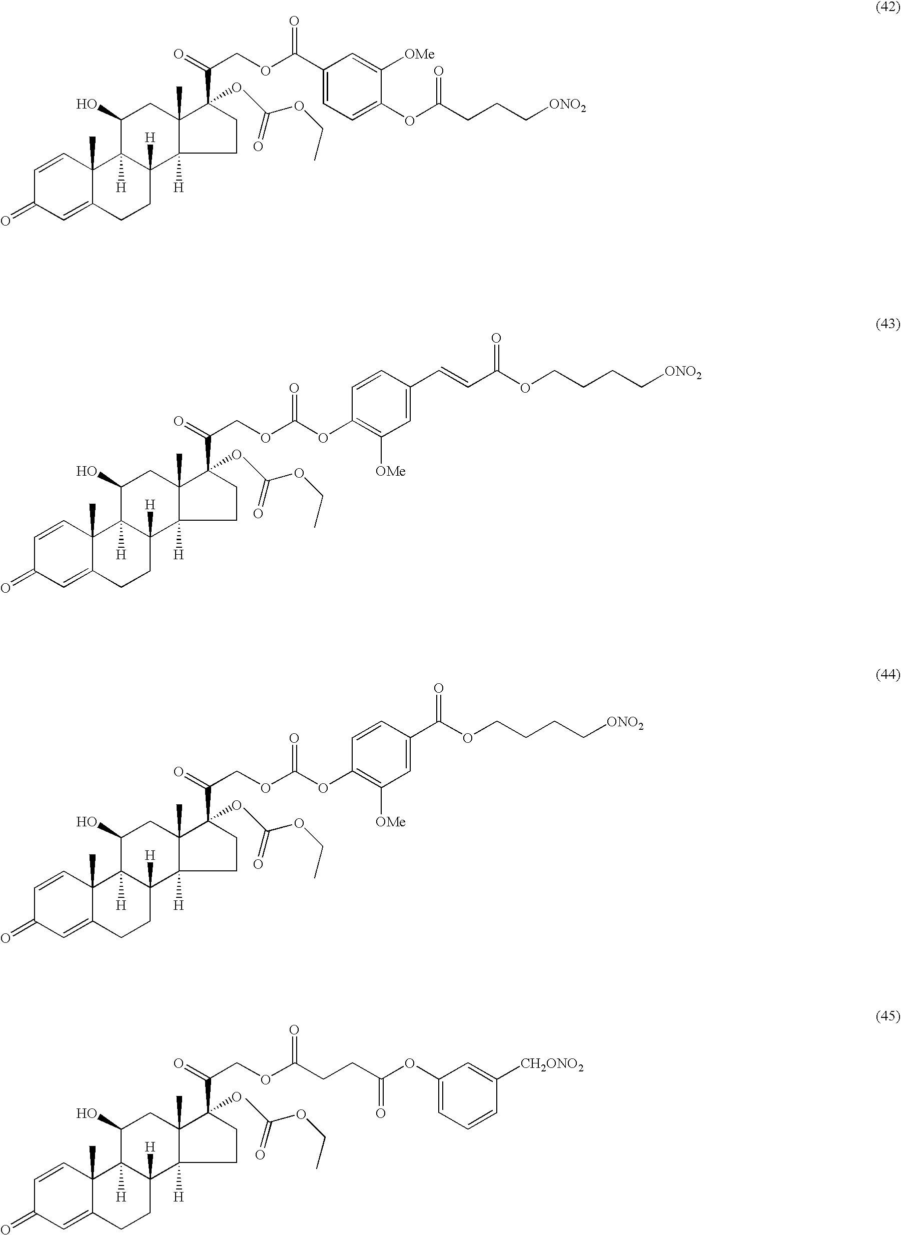

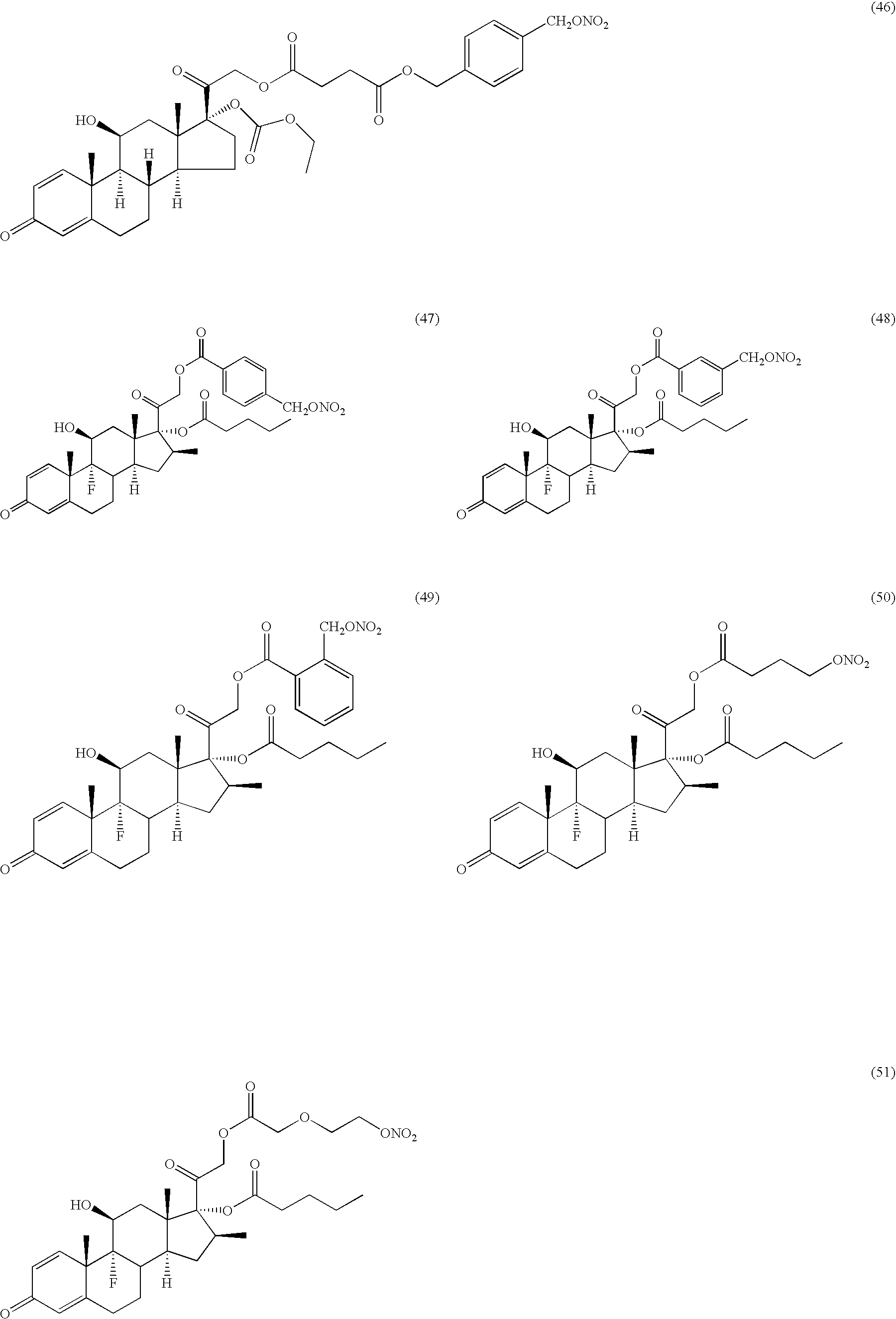

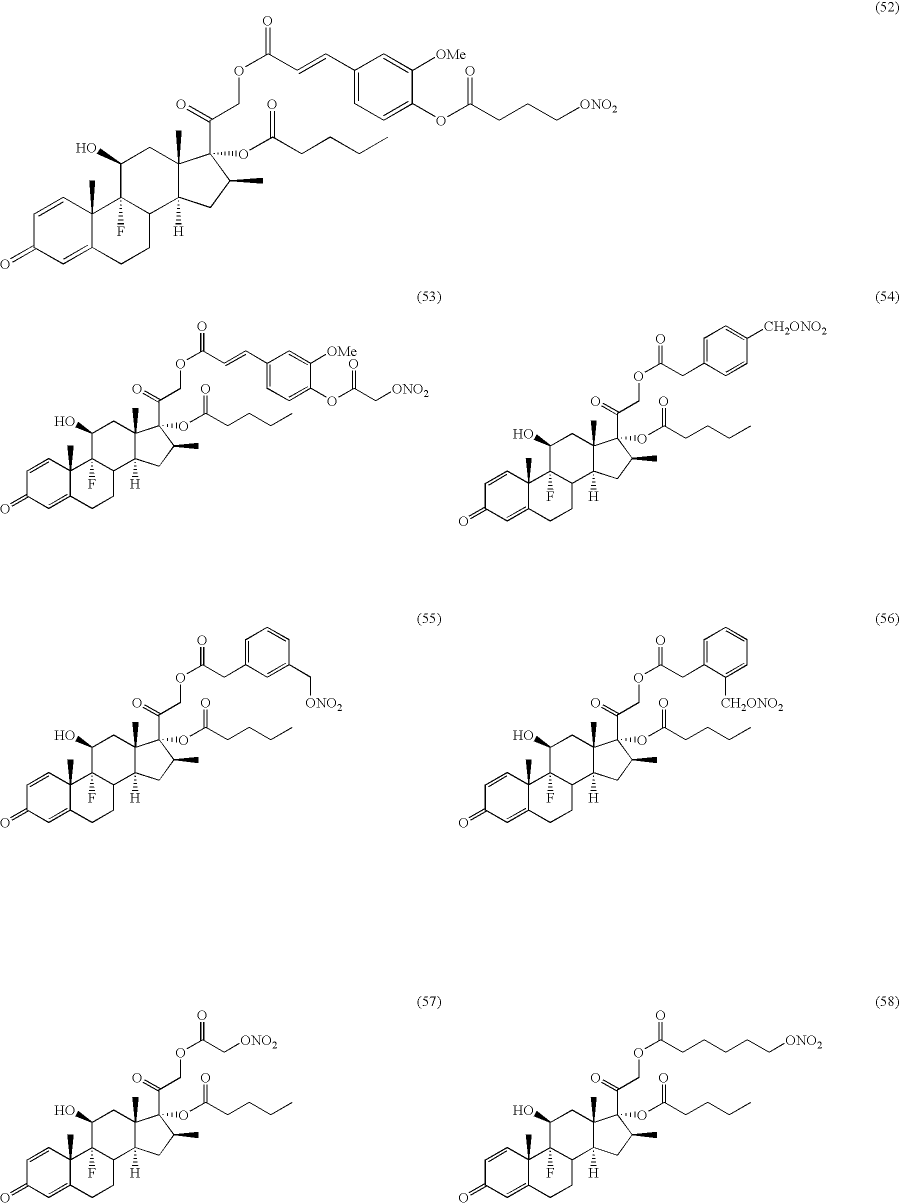

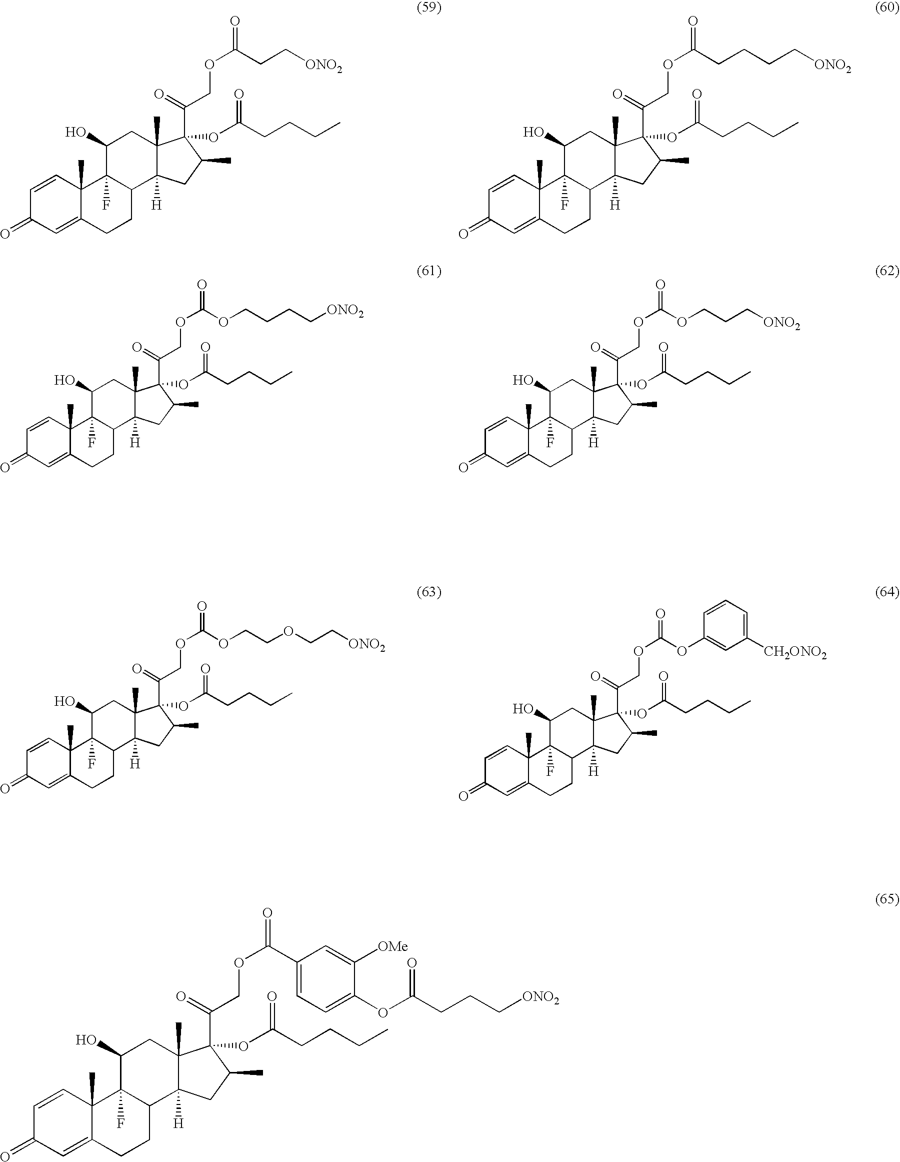

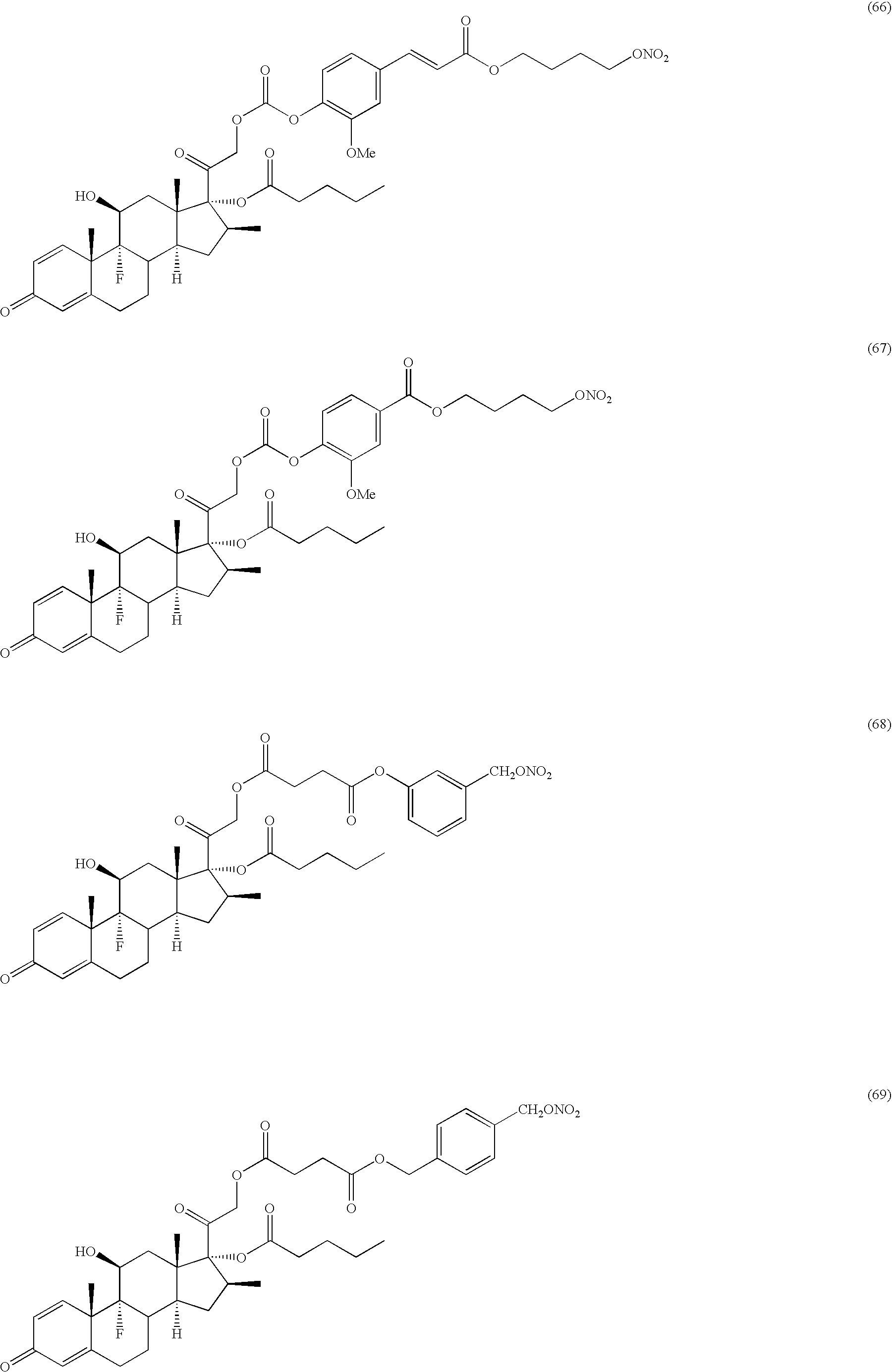

Another embodiment of the present invention is the use of compounds of formula (I)

R-Z-X—ONO₂ (I)

for treating skin or mucosal membrane diseases or disorders, wherein in formula (I):

R is the corticosteroid residue of formula (II):

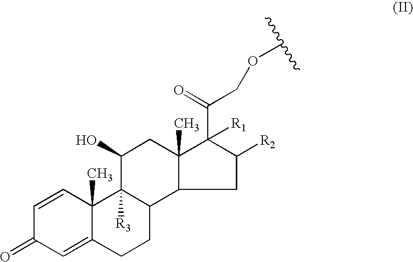

wherein
R₁ is —OC(O)OₘRᵢ wherein m is 0 or 1, Rᵢ is a branched or straight C₁-C₁₀ alkyl, preferably Rᵢ is a branched or straight C₁-C₆ alkyl, preferred Rᵢ groups are; methyl, ethyl, n-propyl, n-butyl;
R₂ is an hydrogen atom or —CH₃;
or R₁ and R₂ when taken together are the group of formula (III)

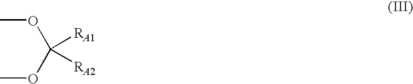

wherein R_{A1} and R_{A2} are independently selected from H, a C₁-C₁₀ linear or branched alkyl chain, preferably (C₁-C₈) alkyl; more preferably R_{A1} and R_{A2} are —CH₃ and the group of formula (III) is a isopropylidenedioxy;
R₁ and R₂ can be linked to the carbon atoms in 16 and 17 of the steroidal structure in position α or β;
R₃ is a hydrogen atom or a fluorine atom;
preferably in formula (II) R₁, R₂ and R₃ have the following meanings:

R₁ and R₂ both in position α are taken together and forms the group of formula (III) wherein R_{A1} and R_{A2} are —CH₃, R₃ is a fluorine atom; or

R₁ is —OC(O)OₘRᵢ in position α wherein m is 1 and Rᵢ is ethyl, R₂ and R₃ are hydrogen atoms; or

R₁ is —OC(O)OₘRᵢ in position α wherein m is 0, Rᵢ is n-butyl, R₂ is —CH₃ in position β, R₃ is a fluorine atom;
Z is a group capable of binding X selected from the group consisting of:

### —C(O)—, —C(O)O— or

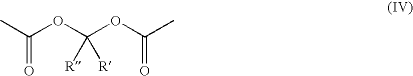

wherein R′ and R″ are independently selected from H or straight or branched C₁-C₄ alkyl; preferably Z is —C(O)— or —C(O)O—;
X is a bivalent radical having the following meanings:
c) straight or branched C₁-C₂₀ alkylene, preferably a straight or branched C₁-C₁₀ alkylene, being optionally substituted with one or more of the substituents selected from the group consisting of: halogen atoms, hydroxy, —ONO₂ or T, wherein T is —OC(O)(C₁-C₁₀ alkyl)-ONO₂ or —O(C₁-C₁₀ alkyl)-ONO₂; preferably X is a straight C₁-C₁₀ alkylene;
d) a C₅-C₇ cycloalkylene group optionally substituted with linear or branched C₁-C₁₀ alkyl group, preferably CH₃;
c)

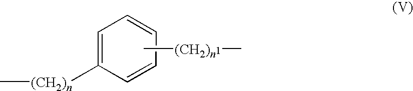

d)

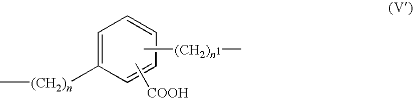

wherein n is an integer from 0 to 20, preferably n is an integer from 0 to 5; more preferably n is 0 or 1;
n¹ is an integer from 1 to 20, preferably n¹ is an integer from 1 to 5; more preferably n¹ is 1;
e)

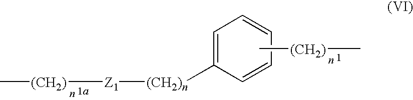

wherein n^{1a} is an integer from 1 to 20, preferably n^{1a} is an integer from 1 to 10;
Z₁ is —C(O)O— or —OC(O)—; preferably Z₁ is —C(O)O—;
n is as above defined;
n¹ is as above defined;
preferably in formula (VI) n^{1a} is an integer from 1 to 10;
Z₁ is —C(O)O—, n is 0 or 1 and n¹ is 1;
with the proviso that when X is selected from the bivalent radicals mentioned under c)-e), the —ONO₂ group of formula (I) is linked to the —(CH₂)ₙ¹— group;
f)

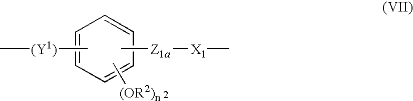

wherein:
Y¹ is —CH₂—CH₂—(CH₂)ₙ^{2a}—, or —CH═CH—(CH₂)ₙ^{2a}— wherein and n^{2a} is an integer from 0 to 10; preferably n^{2a} is 0 or is an integer from 1 to 6;

### Z^{1a} is —OC(O)— or —C(O)O;

n² is 0 or 1; preferably n² is 1;
R² is H or CH₃; preferably R² is CH₃;
X₁ is —(CH)ₙ^{1a}— wherein n^{1a} is as above defined, or the bivalent radical of formula (V) wherein n and n¹ are as above defined;
preferably in formula (VII) Y¹ is —CH═CH—(CH₂)ₙ^{2a}— wherein n^{2a} is 0, Z₁ₐ is —OC(O)—, n² is 1, R² is CH₃, X₁ is —(CH)ₙ^{1a}—, wherein n^{1a} is an integer from 1 to 10;
with the proviso that in formula (VII) the —ONO₂ group of formula (I) is linked to the X₁ group;
g)

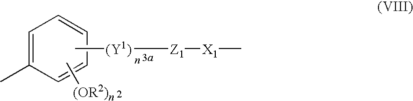

wherein:
Y¹ is —CH₂—CH₂—(CH₂)ₙ^{2a}—, or —CH═CH—(CH₂)ₙ^{2a}— wherein and n is an integer from 0 to 10; preferably n^{2a} is 0 or n^{2a} is an integer from 1 to 6;
n^{3a} is 0 or 1;

### Z₁ is —C(O)O— or —OC(O)—;

n² is 0 or 1; preferably n² is 1;
R² is H or CH₃; preferably R² is CH₃;
X₁ is —(CH)ₙ^{1a}— wherein n^{1a} is as above defined, or the bivalent radical of formula (V) wherein n and n¹ are as above defined;
preferably in formula (VIII) n^{3a} is 1, Y¹ is —CH═CH—(CH₂)ₙ^{2a}— wherein n² is 0, Z₁ is —C(O)O—, n² is 1, R² is CH₃, X₁ is —(CH)ₙ^{1a}— wherein n^{1a} is an integer from 1 to 10; or in formula (VIII) n^{3a} is 0, Z₁ is —OC(O)— or —C(O)O—, n² is 1, R² is CH₃ and X₁ is —(CH)ₙ^{1a}— wherein n^{1a} is an integer from 1 to 10;
with the proviso that in formula (VIII) the —ONO₂ group of formula (I) is linked to the X₁ group;
h)

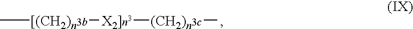

wherein

### X₂ is —O— or —S—;

n³ is an integer from 1 to 6, preferably from 1 to 4, and
n^{3b} is an integer from 1 to 10, preferably from 1 to 6, more preferably n^{3b} is 1 or 2;
n^{3c} is an integer from 1 to 10, preferably from 1 to 6, more preferably n^{3c} is 2;
i)

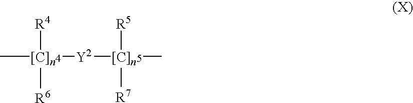

wherein:
n⁴ is an integer from 0 to 10;
n⁵ is an integer from 1 to 10;
R⁴, R⁵, R⁶, R⁷ are the same or different, and are H or straight or branched C₁-C₄ alkyl, preferably R⁴, R⁵, R⁶, R⁷ are H;
wherein the —ONO₂ group of formula (I) is linked to

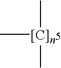

wherein n⁵ is as defined above;
Y² is an heterocyclic saturated, unsaturated or aromatic 5 or 6 members ring, containing one or more heteroatoms selected from nitrogen, oxygen, sulfur,
and is selected from

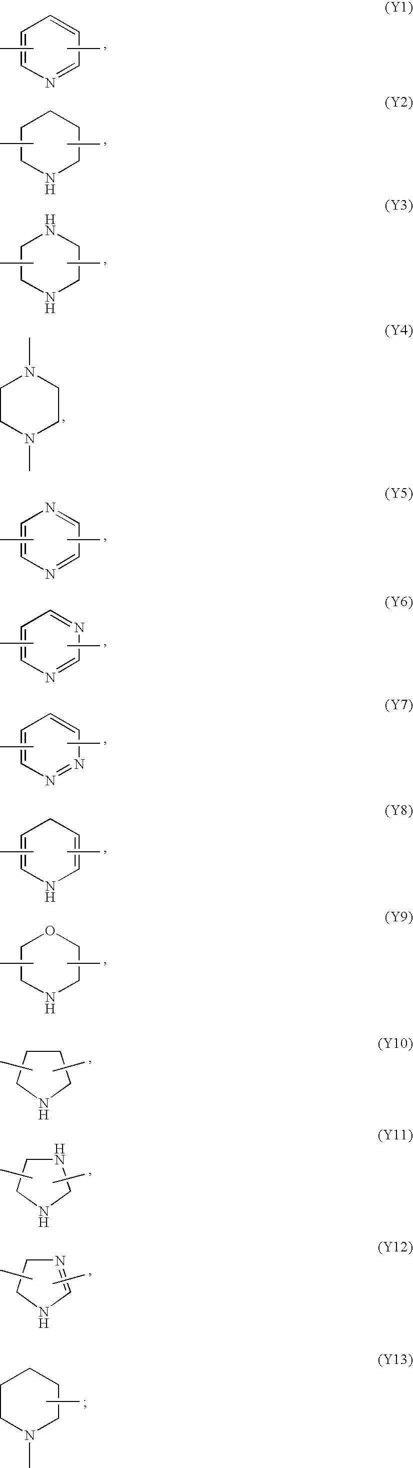

The most preferred compounds of formula (I), above reported, that can be used for treating skin or mucosal membrane diseases or disorders are:

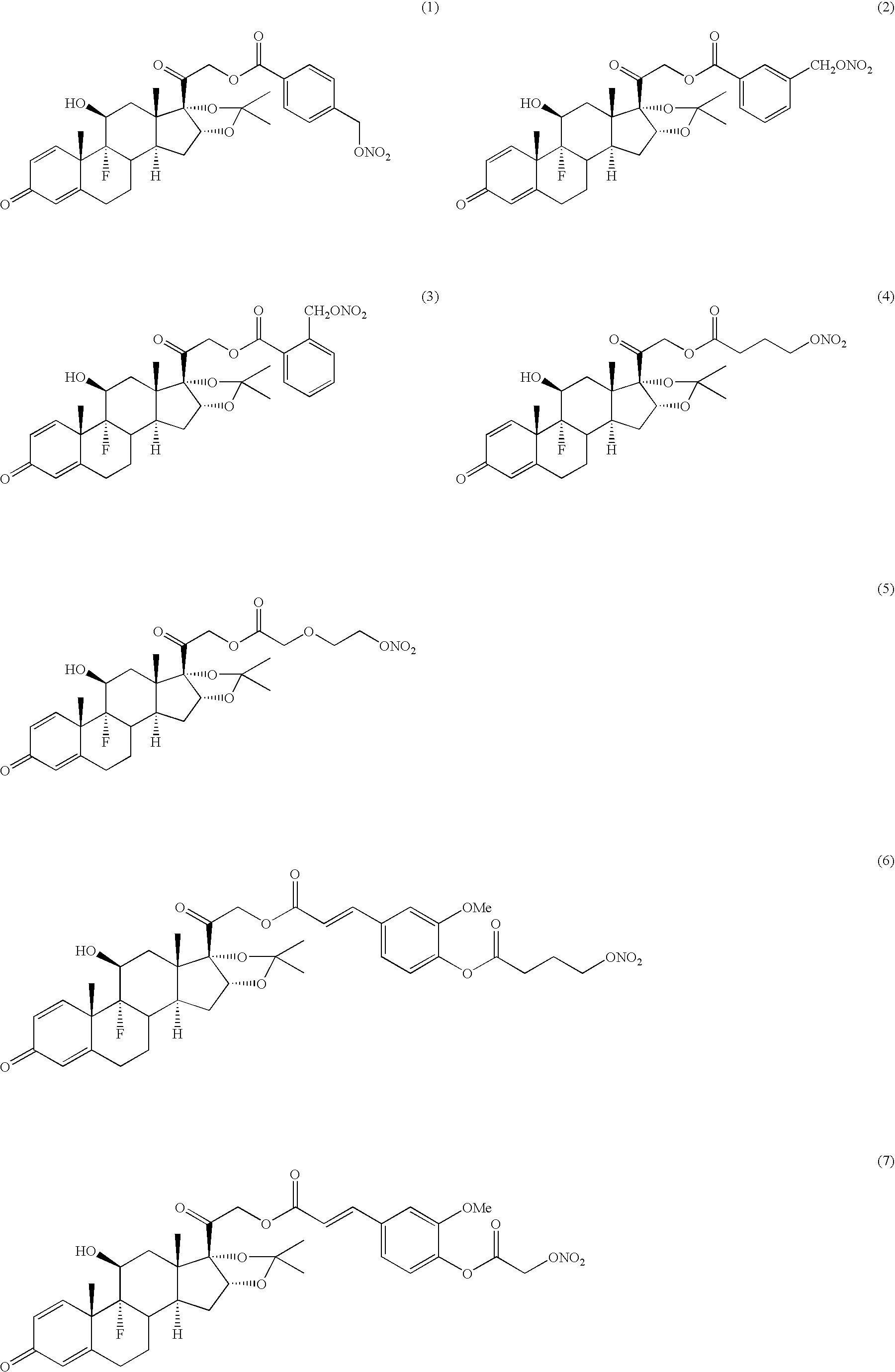

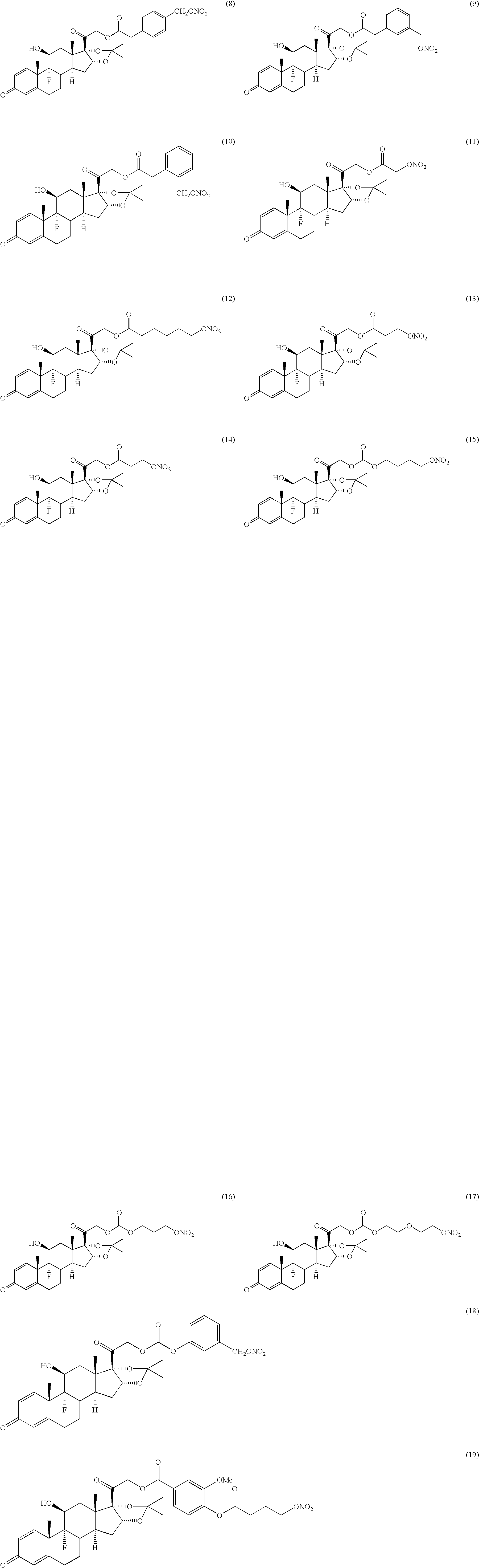

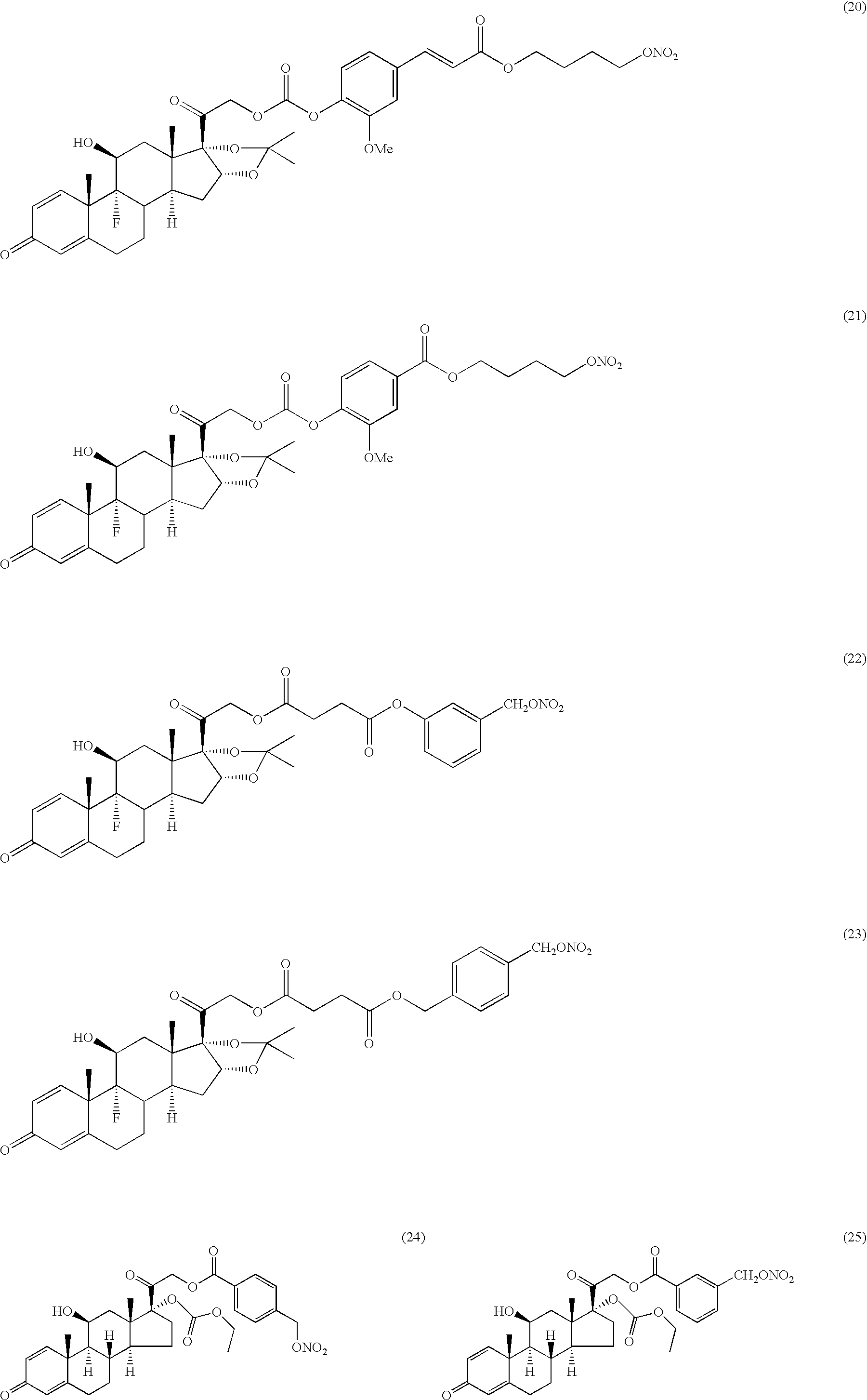

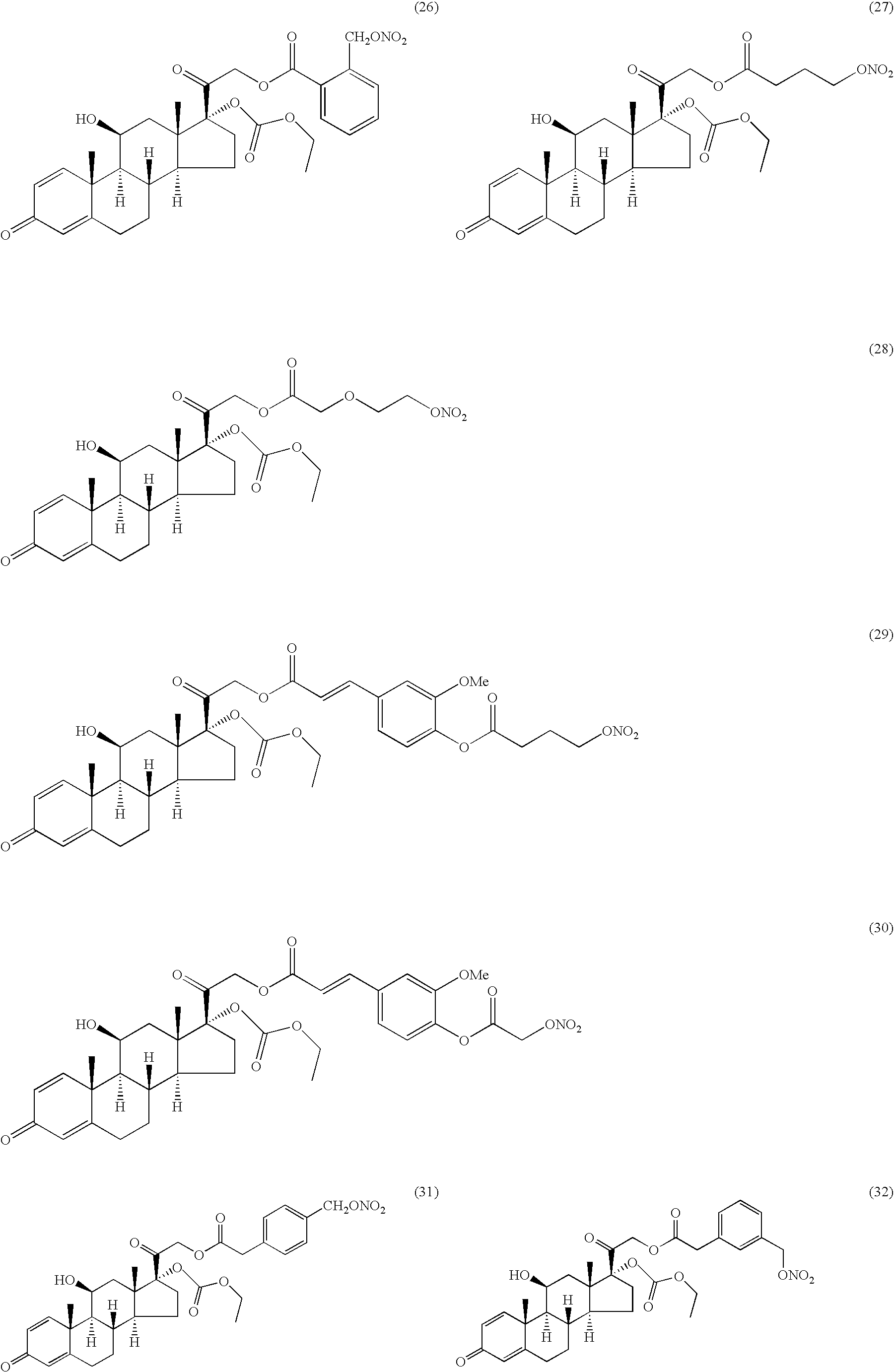

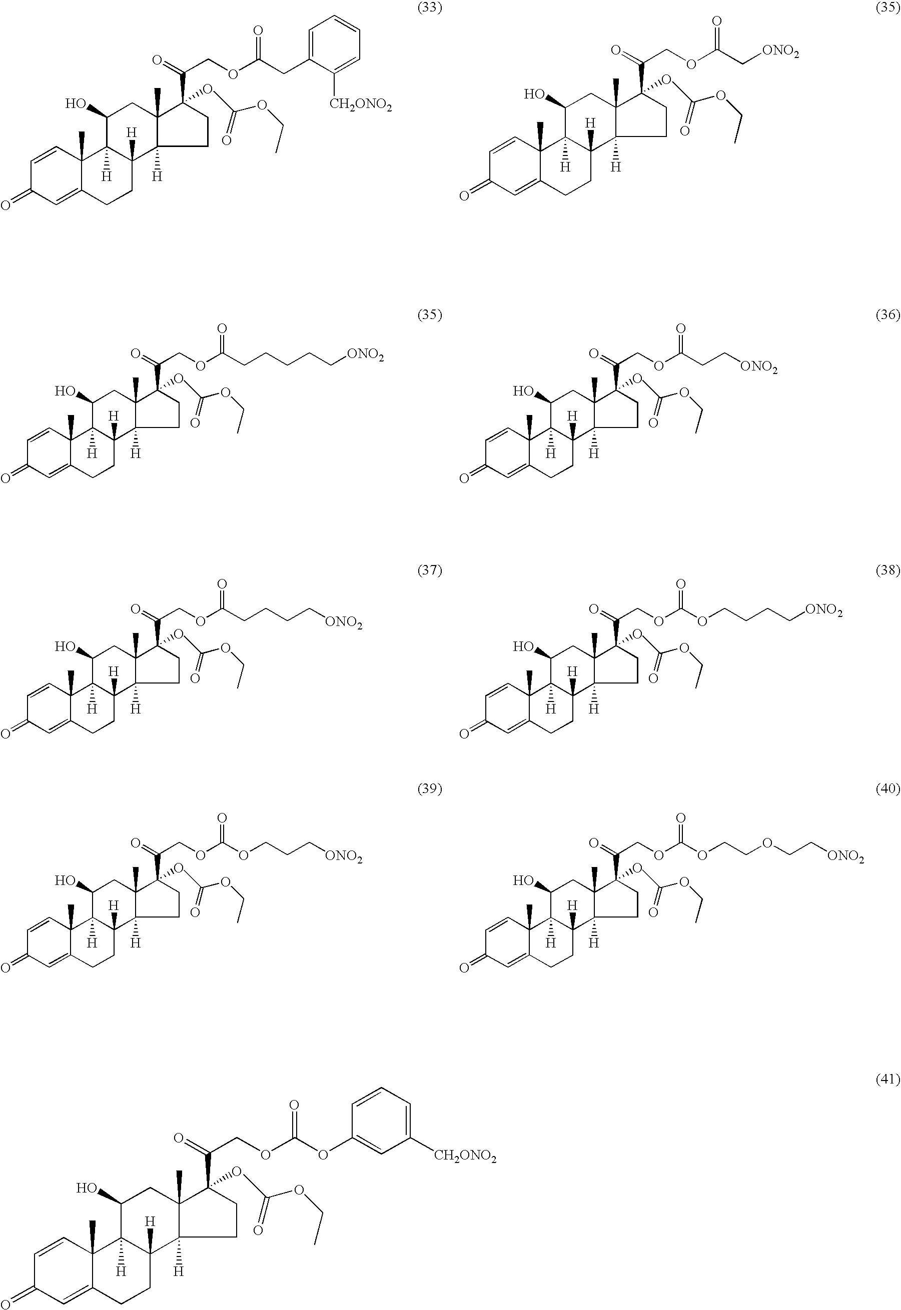

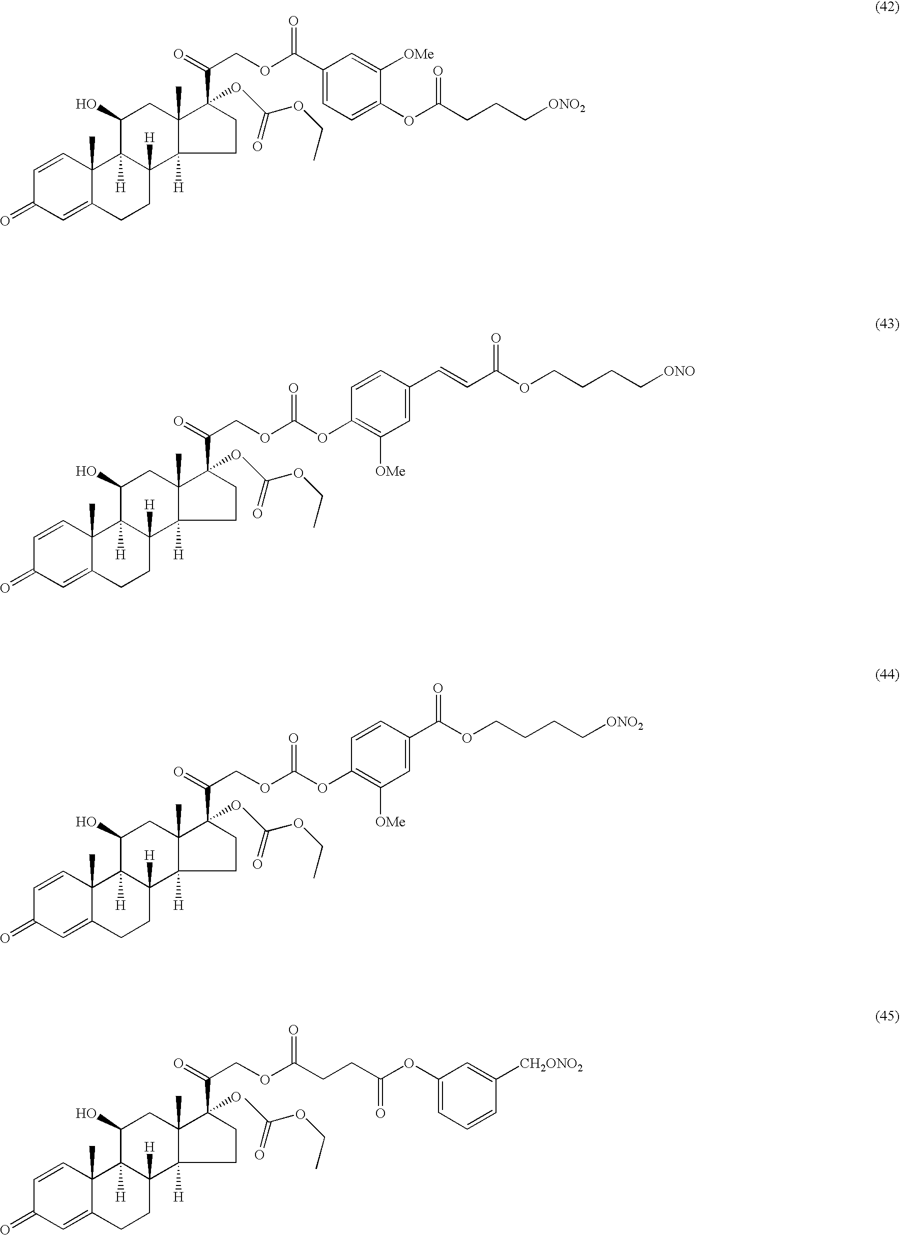

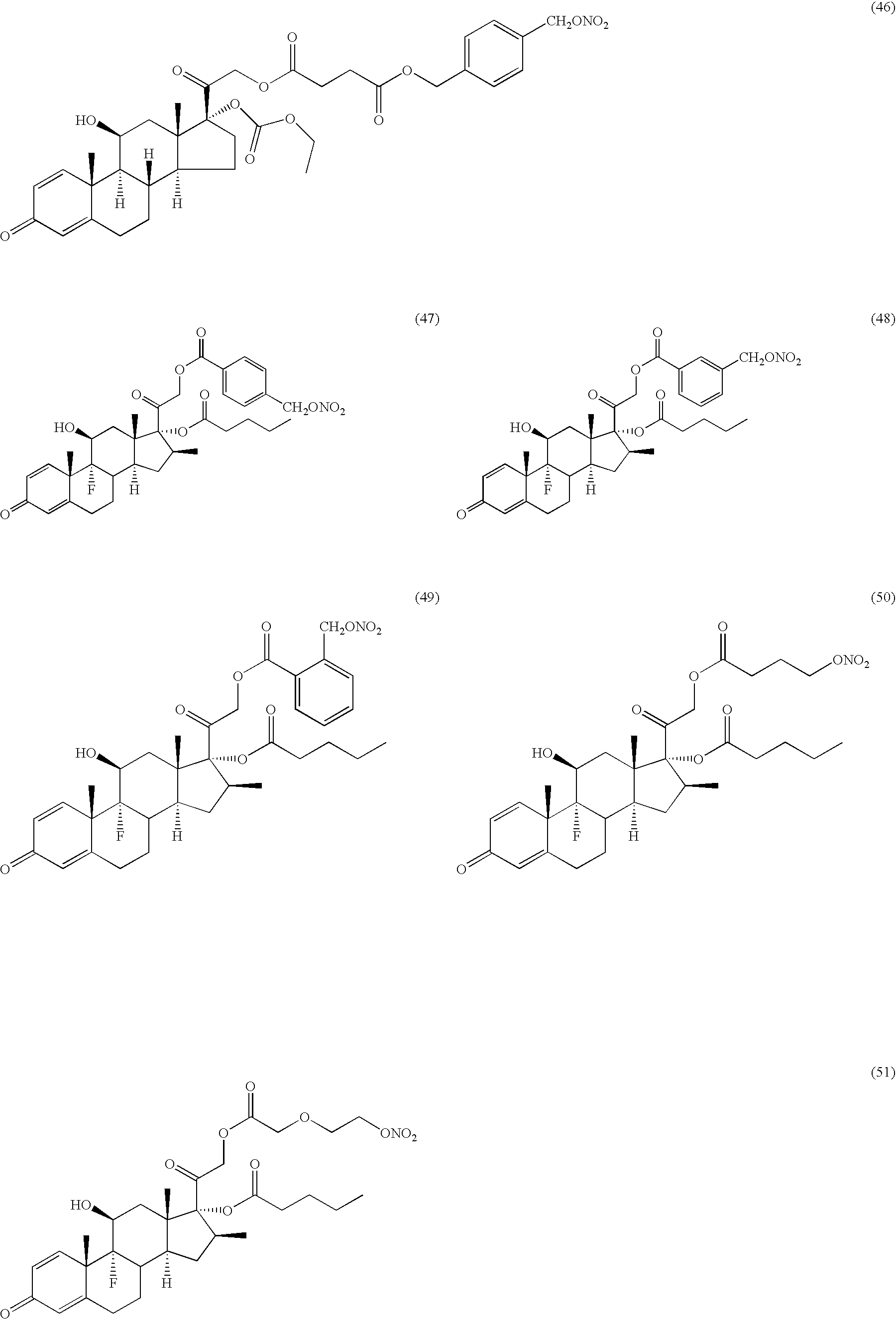

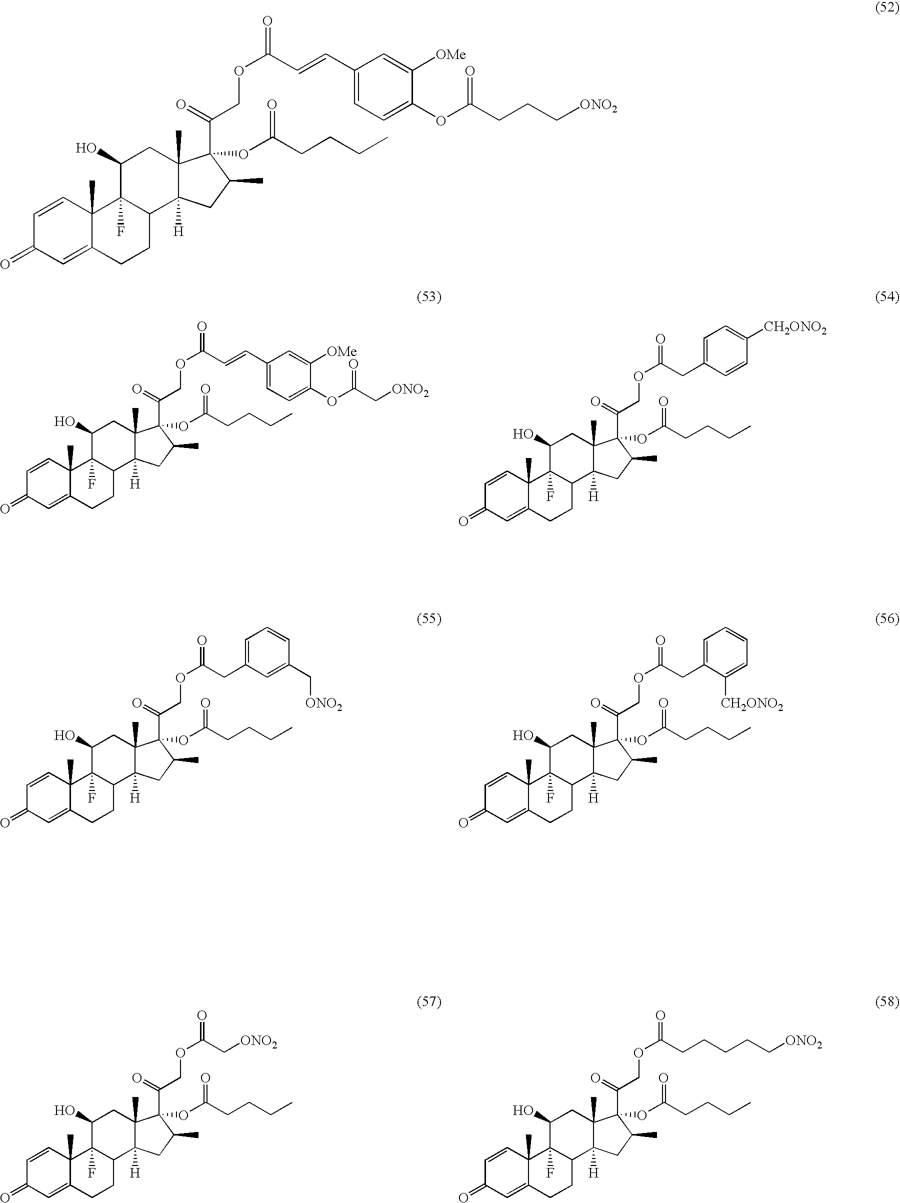

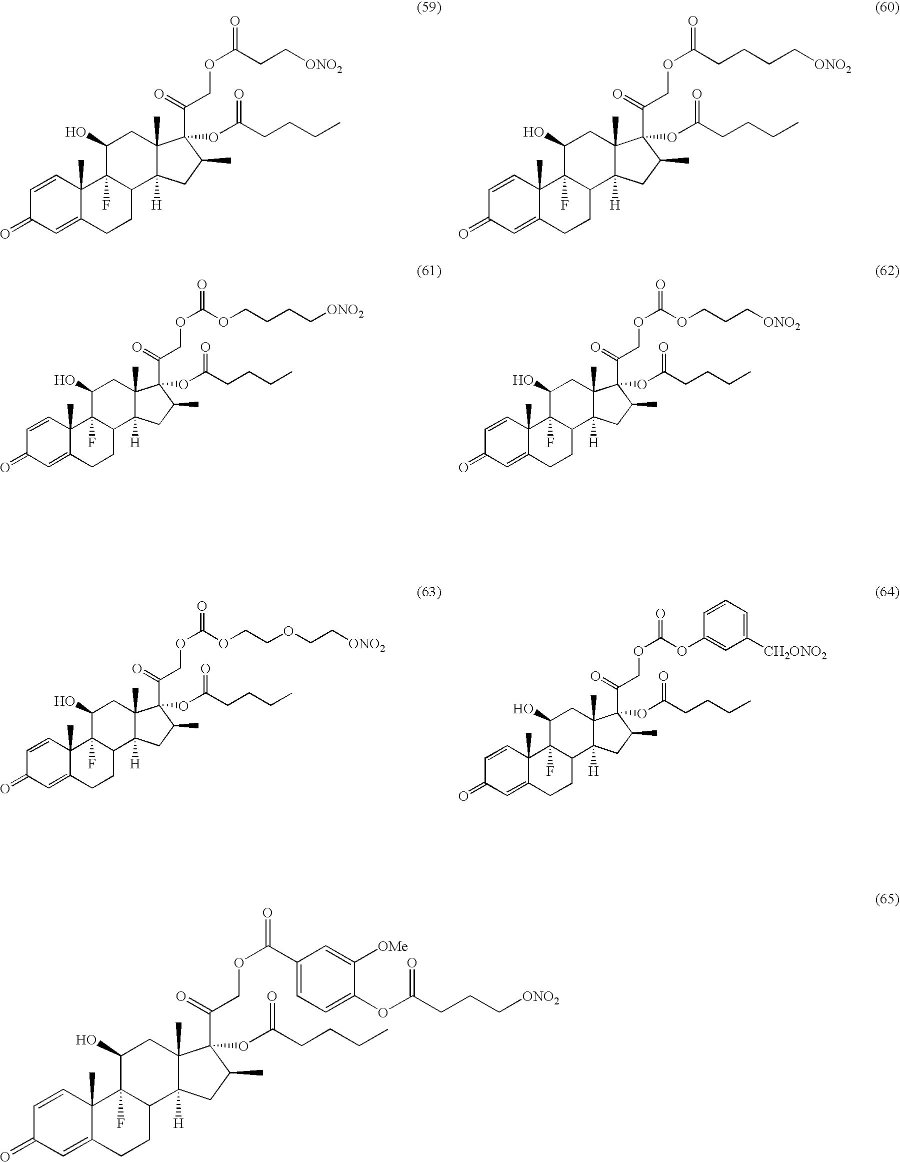

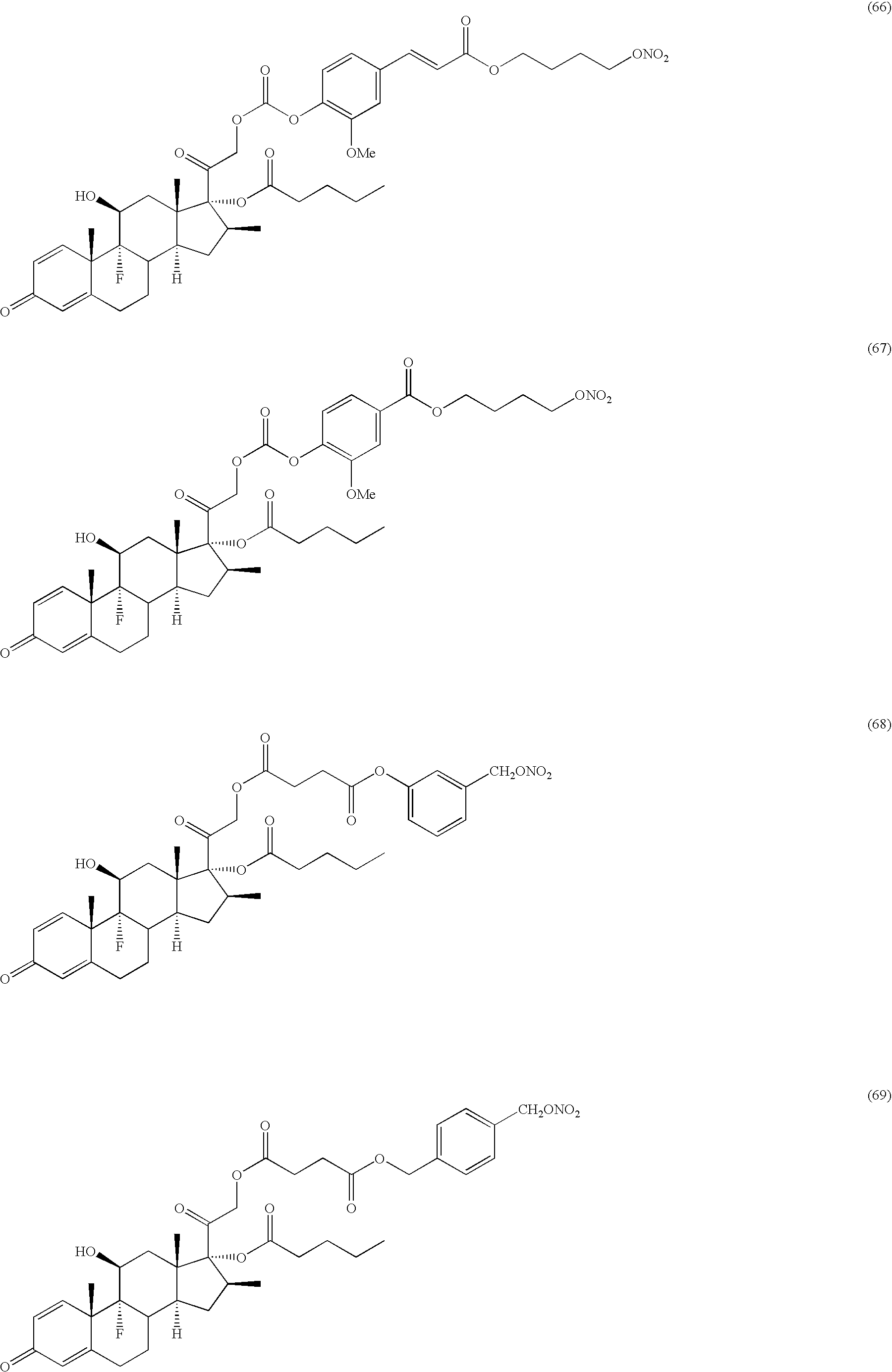

The compounds of the present invention are useful for the treatment of skin or mucosal membrane diseases or disorders comprise, but not limited, corticosteroid-responsive dermatosis, atopic dermatitis, contact dermatitis, seborrheic dermatitis, inflammation, eczema, erythema, papulation, scaling, erosion, oozing, crusting, pruritis, psoriasis, epidermalysis bullosa, erythema, hidradenitis suppurative, warts, diaper rash, jock itch, ruber lichen planus, seborrheic dermatitis which affects the scalp and other hairy areas.

The compounds of the present invention are particularly useful for the treatment of corticosteroid-responsive dermatosis, atopic dermatitis, contact dermatitis, psoriasis, seborrheic dermatitis.

The term “C₁-C₂₀ alkylene” as used herein refers to branched or straight C₁-C₂₀ hydrocarbon chain, preferably having from 1 to 10 carbon atoms such as methylene, ethylene, propylene, isopropylene, n-butylene, pentylene, n-hexylene and the like.

The term “C₁-C₁₀ alkyl” as used herein refers to branched or straight alkyl groups comprising 1 to 10 carbon atoms, including methyl, ethyl, n-propyl, isopropyl, n-butyl, isobutyl, t-butyl, pentyl, hexyl, octyl and the like.

The term “heterocyclic” as used herein refers to saturated, unsaturated or aromatic 5 or 6 members ring, containing one or more heteroatoms selected from nitrogen, oxygen, sulphur, such as for example pyridine, pyrazine, pyrimidine, pyrrolidine, morpholine, imidazole and the like.

This invention includes also the pharmaceutically acceptable salts of the compounds of formula (I), stereoisomers and epimers thereof.

Examples of pharmaceutically acceptable salts are either those with inorganic bases, such as sodium, potassium, calcium and aluminium hydroxides, or with organic bases, such as lysine, arginine, triethylamine, dibenzylamine, piperidine and other acceptable organic amines.

Examples of organic acids are: oxalic, tartaric, maleic, succinic, citric acids. Examples of inorganic acids are: nitric, hydrochloric, sulphuric, phosphoric acids. Salts with nitric acid are preferred.

The compounds of the invention which have one or more asymmetric carbon atoms can exist as optically pure enantiomers, pure diastereomers, enantiomers mixtures, diastereomers mixtures, enantiomer racemic mixtures, racemates or racemate mixtures. Within the scope of the invention are also all the possible isomers, stereoisomers and their mixtures of the compounds of formula (I), including mixtures enriched in a particular isomer.

Skin or mucosal membrane diseases or disorders comprise, but not limited, corticosteroid-responsive dermatosis, atopic dermatitis, contact dermatitis, seborrheic dermatitis, inflammation, eczema, erythema, papulation, scaling, erosion, oozing, crusting, pruritis, psoriasis, epidermalysis bullosa, erythema, hidradenitis suppurative, warts, diaper rash, jock itch, ruber lichen planus.

Also within the scope of the invention are pharmaceutical formulations suitable for topical administration comprising at least a compound of formula (I) of the present invention.

Preferred pharmaceutical dosage forms include cream, lotion and ointment formulation or topical spray compositions.

The pharmaceutical dosage forms are prepared according to procedures well known in the art.

The proportion of the active component of formula (I) in the topical formulation according to the invention depends on the precise type of formulation to be prepared but will generally be within the range of around 0.001-12% by weight, more preferably 0.001 to 10% by weight. Generally, however for most types of preparations advantageously the proportion used will be within the range of from 0.001 to 1% by weight, more preferably 0.01-0.5%, and especially around 0.025 to 0.1%.

Various optional ingredients may also be present in the topical formulations. These are: one or more various solvents such as various short chain alcohols including, but not limited to, ethyl alcohol, propylene glycol, triacetin, hexylene glycol, and combinations thereof; suitable occlusive agents that may be present in the topical formulation include, but are not limited to, petrolatum, microcrystalline wax, dimethicone, beeswax, mineral oil, squalane, liquid paraffin, shea butter, carnauba wax, SEPIGEL® (a blend of isoparaffin/polyacrylamide-/laureth-7), and combinations thereof; surfactant such as, but are not limited to, CETOMACROGOL® 1000, (Crodor, Inc.) glycerol monostearate, glycerol distearate, glyceryl stearate, polyoxyethylene stearate, a blend of glyceryl stearate and PEG-100 stearate (as ARLACEL 165), polysorbate 40, polysorbate 60, polysorbate 80, CETETH-20®, sorbitan monopalimate, sorbitan monostearate, sorbitan monooleate, and combinations thereof. Other various optional ingredients may also be present in the topical formulation. These are carriers (such as water or mineral oil), skin conditioners (such as lanolin, glycerine, cholesterol, cetostearyl alcohol, dimethicone PEG 100, PEG 200, PEG 300, PEG 400 or isopropylmyristate), buffers (such as sodium citrate/citric acid, dibasic sodium phosphate/citric acid, or monobasic sodium phosphate/citric acid), or preservatives (such as imidurea, methylparaben, or propylparaben).

### EXPERIMENTAL PART

### Synthesis Procedure

The compound of general formula (I) as above defined wherein Z is —CO— and X is as above defined, can be obtained

1a) by reacting a compound of formula (IIa), i.e. the precursor corticosteroid,

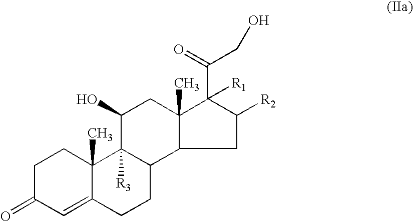

wherein R₁, R₂ and R₃ are as above defined with a compound of formula (Ib)

W—C(O)—X-Q (Ib)

wherein
W is —OH, Cl, or —OC(O)Rₐ wherein Rₐ is a linear or branched C₁-C₅ alkyl or Rₐ is Rₐ₁ selected from the group consisting of: pentafluorophenoxy, 4-nitrophenoxy or succimidinyloxy;
Q is —ONO₂ or Z₂ wherein Z₂ is selected from the group consisting of: a chlorine atom, a bromine atom, a iodine atom, a mesyl group or a tosyl group, in the presence of a condensing agent, and
1b) when Q is Z₂, by converting the compound obtained in the step a) into a nitro derivative by reaction with a nitrate source.

In step 1a) the reaction of a compound of formula (IIa) with the compound of formula (Ib) wherein W is —OH, may be carried out in presence of a condensing agent as dicyclohexylcarbodiimide (DCC), N′-(3-dimethylaminopropyl)-N-ethylcarbodiimide hydrochloride (EDAC) and a catalyst, such as N,N-dimethylamino pyridine (DMAP) or N,N′-carbonyldiimidazole (CDI). The reaction is carried out in an inert organic solvent dry such as N,N′-dimethylformamide, tetrahydrofuran, benzene, toluene, dioxane, a polyhalogenated aliphatic hydrocarbon at a temperature from −20° C. and 40° C. The reaction is completed within a time range from 30 minutes to 36 hours;

In step 1a) the reaction of a compound of formula (IIa) with the compound of formula (Ib) wherein W is —OC(O)Rₐ wherein Rₐ is as above defined, may be carried out in presence of a catalyst, such as N,N-dimethylamino pyridine (DMAP) or in the presence of DMAP and a Lewis acid such as Sc(OTf)₃ or Bi(OTf)₃.

The reaction is carried out in an inert organic solvent such as N,N′-dimethylformamide, tetrahydrofuran, benzene, toluene, dioxane, a polyhalogenated aliphatic hydrocarbon at a temperature from −20° C. and 40° C. The reaction is completed within a time range from 30 minutes to 36 hours. In step 1a) the reaction of a compound of formula (IIa) with the compound of formula (Ib) wherein W is Cl, X is as above defined and Q is Z₂, may be carried out in presence of an organic base such as N,N-dimethylamino pyridine (DMAP), triethylamine, pyridine. The reaction is carried out in an inert organic solvent such as N,N′-dimethylformamide, tetrahydrofuran, benzene, toluene, dioxane, a polyhalogenated aliphatic hydrocarbon at a temperature from −20° C. and 40° C. The reaction is completed within a time range from 30 minutes to 36 hours.

In step 1b) the nitrate source may be silver nitrate, lithium nitrate, sodium nitrate, potassium nitrate, magnesium nitrate, calcium nitrate, iron nitrate, zinc nitrate or tetraalkylammonium nitrate (wherein alkyl is C₁-C₁₀ alkyl). The reaction is carried out in a suitable organic solvent such as acetonitrile, tetrahydrofurane, methyl ethyl ketone, ethyl acetate, DMF, in the dark, at a temperature from room temperature to the boiling temperature of the solvent. The preferred nitrate source is silver nitrate.

The compounds of formula (IIa) are commercially available or can be synthesised as described in the reference documents reported in The Merck Index—Thirteenth Edition. The compound of formula (IIa) above reported wherein R₁ and R₂ both in position α are taken together and forms the group of formula (III) wherein R_{A1} and R_{A2} are —CH₃, R₃ is a fluorine atom is known by generic name of Triamcinolone acetonide.

The compound of formula (IIa) above reported wherein R₁ is —OC(O)OₘRᵢ in position α wherein m is 1 and Rᵢ is ethyl, R₂ and R₃ are hydrogen atoms is known by generic name of prednisolone-17-ethylcarbonate.

The compound of formula (IIa) above reported wherein R₁ is —OC(O)OₘRᵢ in position α wherein m is 0 and Rᵢ is n-butyl, R₂ is —CH₃ in position β, R₃ is a fluorine atom is known by generic name of betamethasone-17-valerate.

The compounds of formula (Ib) wherein Q is OH and X and Z₂ are as above defined, are commercially available or can be synthesized from the corresponding hydroxyl acid of formula HO—C(O)—X—OH by process well known in the art;

The compounds of formula (Ib) wherein Q is ONO₂ may be prepared from the corresponding compounds wherein Q is Z₂ by conversion to the nitro derivative as above described in step 1b).

The compounds of formula (Ib) wherein W=—OC(O)Rₐ and wherein Rₐ, X and Q are as above defined may be obtained from the corresponding acids wherein W=—OH by reaction with a chloroformate such as isobutylchloroformate, ethylchloroformate in presence of a non-nucleophilic base such as triethylamine in an inert organic solvent such as N,N′-dimethylformamide, tetrahydrofuran, a polyhalogenated aliphatic hydrocarbon at a temperature from −20° C. and 40° C. The reaction is completed within a time range from 1 to 8 hours.

The compounds of formula (Ib) wherein W=Cl may be obtained from the corresponding acids wherein W=—OH by reaction with a thionyl or oxalyl chloride, halides of P^{III} or P^{V} in solvents inert such as toluene, chloroform, DMF.

The compound of general formula (I) as above defined wherein Z is —C(O)O— and X is as above defined, can be synthesised

2a) by reacting a compound of formula (IIa) above reported, with a compound of formula (Ic)

R_{b}—C(O)O—X-Q (IC)

wherein X and Q are as above defined, R_{b} is Cl, Br or Rₐ₁ wherein Rₐ₁ is as above defined;
2b) when Q is Z₂, by converting the compound obtained in the step 2a) into the nitro derivative by reaction with a nitrate source as described above.

In step 2a) the reaction is generally carried out in presence of a inorganic or organic base in an aprotic polar/non-polar solvent such as DMF, THF or CH₂Cl₂ at temperatures range between 0°-65° C. or in a double phase system H₂O/Et₂O at temperatures range between 20°-40° C.; or in the presence of DMAP and a Lewis acid such as Sc(OTf)₃ or Bi(OTf)₃ in solvents such as DMF, CH₂Cl₂.

The compound of formula (Ic) wherein X and Q are as above defined and R_{b} is Cl, Br may be synthesized from the corresponding alcohol of formula (Id) HO—X-Q by process well known in the art.

The compounds of formula (Ic) wherein Q is Z₂ are commercially available.

The compounds of formula (Ic) wherein Q is ONO₂ may be prepared from the corresponding compounds wherein Q is Z₁ by conversion to the nitro derivative as above described.

The compound of formula (Ic) R_{b}—C(O)O—Y-Q wherein Y and Q are as above defined, R_{b} is Rₐ₁ may be obtained reacting a compound of formula (Id) HO—X-Q, with a compound of formula (Ic′) R_{b}—C(O)O-Z₂ wherein Z₂ is as above defined. The reaction is generally carried out in presence of a inorganic or organic base in an aprotic polar/non-polar solvent such as DMF, THF or CH₂Cl₂ at temperatures range between 0°-65° C. or in a double phase system H₂O/Et₂O at temperatures range between 20°-40° C.; or in the presence of DMAP and a Lewis acid such as Sc(OTf)₃ or Bi(OTf)₃ in solvents such as DMF, CH₂Cl₂

The compounds of formula (Ic′) wherein R_{b} is Rₐ₁ and Z₂ is as above defined are commercially available.

### Examples

### Example 1

### Synthesis of (11β,16α)-9-fluoro-11-hydroxy-16,17-[1-methyl ethylidenebis(oxy)]-21-[1-oxo-[4-(nitrooxymethyl)benzoxy]]pregna-1,4-diene-3,20-dione

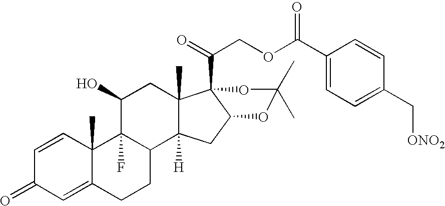

To a solution of triamcinolone acetonide (2.47 g, 5.7 mmol) in dichloromethane (55 ml), 4-(nitrooxymethyl)benzoic acid (1.38 g, 7.0 mmol), DMAP (0.07 g, 0.54 mmol) and EDAC (1.39 g, 7.2 mmol) were added. The reaction was stirred at room temperature for 24 hours. The solution was treated with water, the organic layers were dried with sodium sulfate and concentrated under reduced pressure. The residue was purified by flash chromatography, eluent dichloromethane/ethyl acetate 95/5. The product (1.2 g) was obtained as white powder.

¹H-NMR (DMSO) δ: 8.05 (2H, d); 7.64 (2H, d); 7.29 (1H, d); 6.23 (1H, dd); 6.01 (1H, s); 5.68 (2H, s); 5.52 (1H, d); 5.42 (1H, d); 5.01 (1H, d); 4.86 (1H, d); 4.2 (1H, bs); 2.7-2.25 (4H, m); 2.15-1.72 (4H, m); 1.65-1.45 (5H, m); 1.36 (3H, s); 1.21 (3H, s); 0.87 (3H, s).

### Example 2

### Synthesis of (11β)-17-[(ethoxycarbonyl)oxy]-11-hydroxy-21-[1-oxo-[4-(nitrooxy methyl)benzoxy]]pregna-1,4-diene-3,20-dione

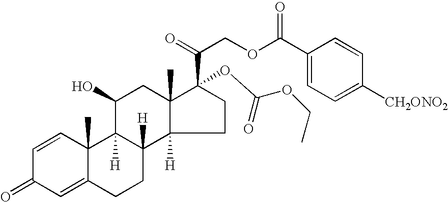

To a solution of prednisolone 17-ethylcarbonate (1.77 g, 4.1 mmol) in dichloromethane (40 ml), 4-(nitrooxymethyl)benzoic acid (1.0 g, 5.0 mmol), DMAP (0.05 g, 0.41 mmol) and EDAC (1.0 g, 5.2 mmol) were added. The reaction was stirred at room temperature for 24 hours. The solution was treated with water, the organic layers were dried with sodium sulfate and concentrated under reduced pressure. The residue was purified by flash chromatography, eluent n-hexane/ethyl acetate 6/4. The product (0.47 g) was obtained as white powder by crystallization with n-hexane/ethylacetate. m.p.=113-119° C.

¹H-NMR (DMSO) δ: 8.07 (2H, d); 7.66 (2H; d); 7.32 (1H, dd); 6.18 (1H, dd); 5.93 (1H, s); 5.70 (2H, s); 5.15 (2H, m); 4.90 (1H, d); 4.33 (1H, m); 4.12 (2H, m); 2.80-2.76 (1H, m); 2.56-2.50 (1H, m); 2.32-2.28 (1H, m); 2.11-1.99 (1H, m); 1.90-1.78 (4H, m); 1.6-1.36 (5H, m); 1.25-1.15 (4H, m); 1.10-0.9 (5H, m).

### Example 3

### Synthesis of (11β,16β)-9-fluoro-11-hydroxy-16-methyl-21-[1-oxo-[4-(nitrooxy methyl)benzoxy]]-17-(valeryloxy)pregna-1,4-diene-3,20-dione

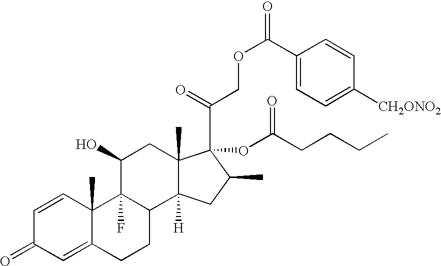

To a solution of betamethasone-17-valerate (2.54 g, 5.3 mmol) in dichloromethane (50 ml), 4-(nitrooxymethyl)benzoic acid (1.3 g, 6.5 mmol), DMAP (0.065 g, 0.53 mmol) and EDAC (1.53 g, 8.0 mmol) were added. The reaction was stirred at room temperature for 4 hours. The solution was treated with water, the organic layers were dried with sodium sulfate and concentrated under reduced pressure. The residue was purified by flash chromatography, eluent n-hexane/ethyl acetate 65/35. The product (0.72 g) was obtained as white powder by crystallization with n-hexane/ethylacetate.

m.p.=158-160° C. ¹H-NMR (DMSO) δ: 8.03 (2H, d); 7.63 (2H, d); 7.29 (1H, d); 6.24 (1H, dd); 6.02 (1H, s); 5.68 (2H, s); 5.6 (1H, d); 4.97 (1H, d); 4.71 (1H, d); 4.24 (1H, m); 2.7-2.2 (4H, m); 2.15-1.75 (6H, m); 1.58-1.05 (13H, m); 0.9 (3H, s); 0.85 (3H, t).

### Example 4

### Synthesis of (11β)-17-[(ethoxycarbonyl)oxy]-11-hydroxy-21-[1-oxo-[3-(nitrooxymethyl)benzoxy]]pregna-1,4-diene-3,20-dione

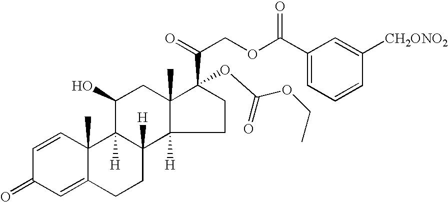

### A) (11β)-17-[(ethoxycarbonyl)oxy]-11-hydroxy-21-[1-oxo-[3-(chloromethyl)benzoxy]]pregna-1,4-diene-3,20-dione

To a solution of prednisolone 17-ethylcarbonate (1.5 g, 3.5 mmol) in dichloromethane (40 ml), 3-(chloromethyl)benzoic acid (0.73 g, 4.2 mmol), DMAP (0.043 g, 0.35 mmol) and EDAC (0.89 g, 4.45 mmol) were added. The reaction was stirred at room temperature for 24 hours. The solution was treated with a saturated solution of sodium bicarbonate, water, the organic layers were dried with sodium sulfate and concentrated under reduced pressure. The residue was purified by flash chromatography, eluent n-hexane/ethyl acetate 1/1. The product (1.32 g) was obtained as white powder.

### B) (11β)-17-[(ethoxycarbonyl)oxy]-11-hydroxy-21-[1-oxo-[3-(nitrooxymethyl)benzoxy]]pregna-1,4-diene-3,20-dione

A solution of compound obtained in step A) (1.3 g, 2.22 mmol) and silver nitrate (0.75 g, 4.44 mmol) in acetonitrile (20 ml) was stirred at 60° C., in the dark, for 10 hours. The precipitated (silver salts) was filtered off and the solvent was evaporated under vacuum. The residue was purified by flash chromatography, eluent n-hexane/ethyl acetate 6/4. The product (1 g) was obtained as white powder.

¹H-NMR (DMSO) δ: 8.11 (1H, m); 8.04 (1H, m); 7.80 (1H, m); 7.62 (1H, m); 7.30 (1H, m); 6.15 (1H, m); 5.91 (1H, s); 5.67 (2H, s); 5.11 (2H, m); 4.88 (1H, bd); 4.31 (1H, bs); 4.11 (2H, m); 2.81-2.71 (1H, m); 2.6-2.49 (1H, m); 2.22-2.32 (1H, m); 2.15-1.95 (2H, m); 1.9-1.7 (4H, m); 1.6-1.3 (4H, m); 1.18 (3H, t); 1.1-0.9 (4H, m).

### Example 5

### Synthesis of (11β,16β)-9-fluoro-11-hydroxy-16-methyl-21-[1-oxo-[3-(nitrooxy methyl)benzoxy]]-17-(valeryloxy)pregna-1,4-diene-3,20-dione

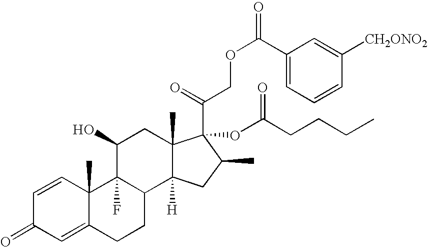

### C) (11β,16β)-9-fluoro-11-hydroxy-16-methyl-21-[1-oxo-[3-(chloromethyl)benzoxy]]-17-(valeryloxy)pregna-1,4-diene-3,20-dione

To a solution of betamethasone 17-valerate (1.5 g, 3.1 mmol) in dichloromethane (35 ml), 3-(chloromethyl)benzoic acid (0.76 g, 4.5 mmol), DMAP (0.038 g, 0.31 mmol) and EDAC (0.77 g, 4.0 mmol) were added. The reaction was stirred at room temperature for 24 hours. The solution was treated with a saturated solution of sodium bicarbonate, water, the organic layers were dried with sodium sulfate and concentrated under reduced pressure. The residue was purified by flash chromatography, eluent n-hexane/ethyl acetate 8/2. The product (1.6 g) was obtained as white powder.

### D) (11β,16β)-9-fluoro-11-hydroxy-16-methyl-21-[1-oxo-[3-(nitrooxymethyl)benzoxy]]-17-(valeryloxy)pregna-1,4-diene-3,20-dione

A solution of compound C (1.68 g, 2.67 mmol) and silver nitrate (1.8 g, 11.2 mmol) in acetonitrile (25 ml) and tetrahydrofurane (2 ml) was stirred at 60° C., in the dark, for 12 hours. The precipitated (silver salts) was filtered off and the solvent was evaporated under vacuum. The residue was purified by flash chromatography, eluent n-hexane/ethyl acetate 6/4. The product (1.1 g) was obtained as white powder.

¹H-NMR (DMSO) δ: 8.11 (1H, s); 8.03 (1H, d); 7.80 (1H, d); 7.62 (1H, t); 7.30 (1H, dd); 6.24 (1H, dd); 6.02 (1H, s); 5.67 (1H, s); 5.58 (1H, d); 4.99 (1H, d); 4.73 (1H, d); 4.29-4.20 (1H, m); 2.59-2.25 (8H, m); 2.19-2.01 (1H, m); 1.89-1.78 (4H, m); 1.56-1.07 (11H, m); 1.0-0.61 (6H, m).

### Example 6

### Synthesis of (11β,16α)-9-fluoro-11-hydroxy-16,17-[1-methylethylidenebis(oxy)]-21-[1-oxo-[3-(nitrooxymethyl)benzoxy]]pregna-1,4-diene-3,20-dione

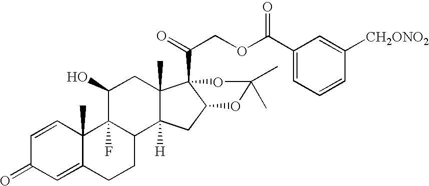

### E) (11β,16α)-9-fluoro-11-hydroxy-16,17-[1-methylethylidene bis(oxy)]-21-[1-oxo-[3-(chloromethyl)benzoxy]]pregna-1,4-diene-3,20-dione

To a solution of triamcinolone acetonide (1.5 g, 3.4 mmol) in dichloromethane (35 ml), 3-(chloromethyl)benzoic acid (0.83 g, 4.9 mmol), DMAP (0.042 g, 0.34 mmol) and EDAC (0.84 g, 4.4 mmol) were added. The reaction was stirred at room temperature for 24 hours. The solution was treated with a saturated solution of sodium bicarbonate, water, the organic layers were dried with sodium sulfate and concentrated under reduced pressure. The product (1.86 g) was obtained as white powder.

### F) (11β,16α)-9-fluoro-11-hydroxy-16,17-[1-methylethylidene bis(oxy)]-21-[1-oxo-[3-(nitrooxymethyl)benzoxy]]pregna-1,4-diene-3,20-dione

A solution of compound E (1.8 g, 3.07 mmol) and silver nitrate (1.1 g, 6.5 mol) in acetonitrile (25 ml) and tetrahydrofurane (10 ml) was stirred at 60° C., in the dark, for 18 hours. The precipitated (silver salts) was filtered off and the solvent was evaporated under vacuum. The residue was purified by flash chromatography, eluent n-hexane/ethyl acetate 65/35. The product (1.3 g) was obtained as white powder

¹H-NMR (DMSO) δ: 8.12 (1H, s); 8.03 (1H, d); 7.80 (1H, d); 7.63 (1H, t); 7.30 (1H, d); 6.23 (1H, dd); 6.01 (1H, s); 5.66 (2H, s); 5.46 (1H, bd); 5.45-5.37 (1H, m); 5.05-4.98 (1H, m); 4.86 (1H, bd); 4.22 (1H, bs); 2.72-2.29 (3H, m); 2.13-1.71 (4H, m); 1.62-1.49 (5H, m); 1.42-1.29 (4H, m); 1.2 (3H, s); 0.9 (3H, s).

### Example 7

### Synthesis of (11β,16α)-9-fluoro-11,21-dihydroxy-16,17-[1-methylethylidenebis(oxy)]pregna-1,4-diene-3,20-dione-21-[3-carboxy-1-oxopropoxy)-3-(nitrooxymethyl)benzene]

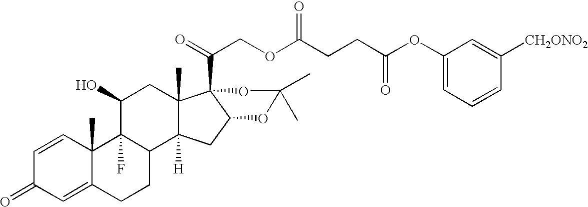

### G) (11β,16α)-9-Fluoro-11,21-dihydroxy-16,17-[1-methyl ethylidenebis(oxy)]pregna-1,4-diene-3,20-dione 21-hemisuccinate

To a solution of triamcinolone acetonide (1.0 g, 2.3 mmol) in tert-butanol (20 ml), succinic anhydride (0.72 g, 7.0 mmol) and triethylamine (0.98 ml, 7.0 mmol) were added. The reaction was stirred at room temperature for 3 hours. The solution was treated with a solution of phosphoric acid 2.5% and dichloromethane, the organic layers were dried with sodium sulfate and concentrated under reduced pressure. The product (1.33 g) was obtained as white powder

### H) (11β,16α)-9-Fluoro-11,21-dihydroxy-16,17-[1-methyl ethylidenebis(oxy)]pregna-1,4-diene-3,20-dione-21-[3-carboxy-1-oxopropoxy)-3-(nitrooxymethyl)benzene]

To a solution of compound obtained in step G (1.23 g, 2.3 mmol) in dichloromethane (50 ml), 3-(nitrooxymethyl)phenol (0.43 g, 2.53 mmol), DMAP (0.028 g, 0.23 mmol) and EDAC (0.56 g, 2.9 mmol) were added. The reaction was stirred at room temperature for 2 hours. The solution was treated with water, the organic layers were dried with sodium sulfate and concentrated under reduced pressure. The residue was purified by flash chromatography, eluent n-hexane/acetone 7/3. The product (1.13 g) was obtained as white powder

¹H-NMR (DMSO) δ: 7.5-7.41 (1H, m); 7.40-7.31 (1H, m); 7.30 (2H, m); 7.19-7.12 (1H, dd); 6.25-6.18 (1H, dd); 6.01 (1H, s); 5.54 (2H, s); 5.43 (1H, dd); 5.22-5.12 (1H, d); 4.85 (1H, d); 4.83-4.73 (1H, m); 4.20 (1H, bs); 2.92-2.79 (4H, m); 2.72-2.24 (4H, m); 2.15-1.72 (4H, m); 1.65-1.45 (5H, m); 1.36 (3H, s); 1.21 (3H, s); 0.87 (3H, s)

### Example 8

### Synthesis of (11β)-17-[(ethoxycarbonyl)oxy]-11,21-dihydroxy pregna-1,4-diene-3,20-dione-21-[3-carboxy-1-oxopropoxy)-3-(nitrooxymethyl)benzene]

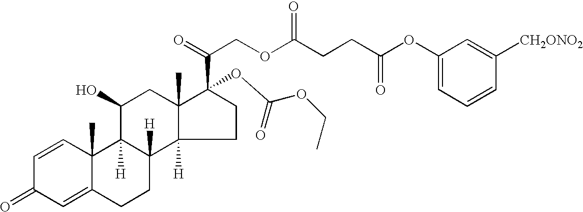

### I) (11β)-17-[(ethoxycarbonyl)oxy]-11,21-dihydroxypregna-1,4-diene-3,20-dione 21-hemisuccinate

To a solution of prednisolone 17-ethylcarbonate (1.1 g, 2.54 mmol) in tert-butanol (22 ml), succinic anhydride (0.77 g, 7.7 mmol) and triethylamine (1.1 ml, 7.75 mmol) were added. The reaction was stirred at room temperature for 3 hours. The solution was treated with a solution of phosphoric acid 2.5% and dichloromethane, the organic layers were dried with sodium sulfate and concentrated under reduced pressure. The product (1.5 g) was obtained.

### L) (11β)-17-[(ethoxycarbonyl)oxy]-11,21-dihydroxypregna-1,4-diene-3,20-dione 21-[3-carboxy-1-oxopropoxy)-3-(nitrooxymethyl)benzene]

To a solution of the compound obtained in step I (1.35 g, 2.5 mmol) in dichloromethane (50 ml), 3-(nitrooxymethyl)phenol (0.473 g, 2.79 mmol), DMAP (0.031 g, 0.25 mmol) and EDAC (0.62 g, 3.2 mmol) were added. The reaction was stirred at room temperature for 4 hours. The solution was treated with water; the organic layers were dried with sodium sulfate and concentrated under reduced pressure. The residue was purified by flash chromatography, eluent n-hexane/acetone 7/3. The product (0.7 g) was obtained as white powder.

¹H-NMR (DMSO) δ: 7.47 (1H, t); 7.36 (1H, d); 3.29 (1H, d); 7.24 (1H, m); 7.16 (1H, m); 6.15 (1H, dd); 5.91 (1H, s); 5.57 (2H, s); 4.84 (2H, m); 4.81 (1H, d); 4.29 (1H, bs); 4.10 (2H, m); 2.86 (2H, m); 2.82 (2H, m); 2.74 (1H, m); 2.6-2.49 (1H, m); 2.28 (1H, m); 2.15-1.92 (2H, m); 1.90-1.61 (4H, m); 1.47 (1H, m); 1.35 (3H, s); 1.15 (3H, t) 1.1-0.9 (1H, m); 0.91 (3H, s).

### Pharmacological Examples

### Example 9

### In Vivo Determination of Inhibition of TPA-Induced Ear Oedema after Topical Administration of Test Compounds

The tested compounds are:
(11β,16α)-9-fluoro-11-hydroxy-16,17-[1-methylethylidene bis(oxy)]-21-[1-oxo-[4-(nitrooxymethyl)benzoxy]]pregna-1,4-diene-3,20-dione, prepared as described in example 1;
Triamcinolone acetonide that is the reference compound for the compound of example 1;
(11β)-17-[(ethoxycarbonyl)oxy]-11-hydroxy-21-[1-oxo-[4-(nitrooxy methyl)benzoxy]]pregna-1,4-diene-3,20-dione, prepared as described in example 2;
Prednicarbate that is the reference compound for the compound of example 2;
   The tests were performed according to the methods described by Carlson et al., Agents Actions 17:197-204, 1985, and Lucas et al., J Pharmacol Exp Ther 304:1172-1180, 2003.

Groups of 5-9 male Swiss mice of 27±5 g were used. Inflammation dermatitis was induced by applying 2 μg/ear of TPA (Tetradecanoyl Phorbol Acetate) dissolved with ethanol absolute on the surface of either the dorsal aspect of both ears (20 μL/ear).

15 min before the application of TPA, mice received topically 20 μL of a solution of test compounds (0.39 nM in ethanol) per site applied directly on the skin of the left ear and the vehicle (ethanol 100%) on right ear. Vehicle-Vehicle treated mice were included as negative control group. Compounds were tested at equimolecular doses.

Animals were sacrificed at 3 h or 5 h post TPA dose. Equal sections of both ears were punched out immediately after and weighed. The percentage of change of the weight of left ear versus the weight of right ear was calculated for each animal, and the percentage of inhibition of change in weight of treated animals versus the change in weight of non-treated animals (negative control) was measured. The results of this test are given in Table 1.

TABLE 1

| | % inhibition | % inhibition |
|---|---|---|
| | 3 hours | 5 hours |
| Compound | post-treatment | post-treatment |
| Compound of Ex. 1 | 22.70 | 21.01 |
| Triamcinolone acetonide | 14.90 | 12.19 |
| Compound of ex. 2 | 33.26 | — |
| Prednicarbate | 19.58 | — |

### Example 10

### Effects of Test Compounds on the 12-O-tetradecanoylphorbol Acetate (TPA)-Induced Increase of Inflammatory Markers (PGE₂ and TNF-α) in Mouse Ear

The tested compounds are:
(11β,16α)-9-fluoro-11-hydroxy-16,17-[1-methylethylidene bis(oxy)]-21-[1-oxo-[4-(nitrooxymethyl)benzoxy]]pregna-1,4-diene-3,20-dione, prepared as described in example 1;
Triamcinolone acetonide that is the reference compound for the compound of example 1;

Ten Swiss male mice were used for treatment group. 12-O-Tetradecanoylphorbol acetate (2.0 μg, TPA) dissolved in 20 μl ethanol absolute was applied in 10 μl volumes to both inner and outer surfaces of the right ear of mice. A ear section of the right ear of mice were homogenized in 500 μl saline, and after centrifugation at 1,200 g for 15 min at 4° C., the PGE₂ and TNF-α levels were determined by radioimmunoassay (Hoult et al., Methods Enzymol 1994) or by time-resolved fluoroimmunoassay (Pennanen et al., Int J. Immunopharmacol. 1995), respectively. Test compounds dissolved in the vehicle were applied topically 15 min before TPA administration. Results are reported in table 2.

TABLE 2

| | TNFalpha | PGE₂ |
|---|---|---|
| Compound | (pg/ml) | (ng/ml) |
| Vehicle + TPA | 316.2 ± 35.5 | 97.9 ± 6.1 |
| Compound of Example 1 | 149.5 ± 17.8** | 53.2 ± 4.5** |
| (0.48 μg/ear) | | |
| Triamcinolone acetonide | 158.0 ± 24.6** | 43.0 ± 3.8** |
| (1 μg/ear) | | |

Results show Mean ± S.E.M. (n = 10).
**P < 0.01 with respect vehicle-TPA group

Results demonstrated that at low doses, the compound of example 1 of the present invention displayed reduction of PGE₂ level than higher dose of the prior-art compound, the results show that the compound of the invention is more effective in reducing the inflammation than the correspondent parent compound.

Surprisingly, the results show that the compound of example 1 of the present invention inhibits the release of TNF-α in a higher potency than the prior art compound as displayed in table 2, therefore being more effective in reducing inflammation levels.

### Example 11

### Anti-Inflammatory Properties in a Model of Contact Dermatitis Induced by Benzalkonium in Mice

The tested compounds are:
(11β,16α)-9-fluoro-11-hydroxy-16,17-[1-methylethylidene bis(oxy)]-21-[1-oxo-[4-(nitrooxymethyl)benzoxy]]pregna-1,4-diene-3,20-dione, prepared as described in example 1;
Triamcinolone acetonide that is the reference compound for the compound of example 1 (ref. compound);
(11β)-17-[(ethoxycarbonyl)oxy]-11-hydroxy-21-[1-oxo-[4-(nitrooxy methyl)benzoxy]]pregna-1,4-diene-3,20-dione, prepared as described in example 2;

Irritant contact dermatitis was induced by applying 5% benzalkonium chloride (100 μl per site, dissolved in olive oil:acetone, 1:5 v/v) on the dorsal aspect of the two ears. Ear diameter was measured as a parameter for edema formation, before and hourly for 6 hours after benzalkonium chloride application, using an electronic caliper. The last measurement was performed at 8-hours after dermatitis induction.

The tested compounds were applied topically dissolved in ethanol:sterile water (1:1) and applied at a final volume of 100 μl, five minutes after irritant contact dermatitis will be induced.

Ear edema (left ears) value for times 1 to 4 hours after irritant stimuli with benzalkonium chloride are represented in the table 3.

The compound of the present invention (comp. Ex. 1) showed a dose-dependent effect in inhibiting ear edema with a better profile than triamcinolone acetonide, mainly at earlier times.

TABLE 3

| | Time after benzalkonium application (hours) | | | |
|---|---|---|---|---|
| Treatment | 1 | 2 | 3 | 4 |
| Comp. Ex. 1 0.3 | 0.026 ± 0.005 | 0.035 ± 0.008 | 0.053 ± 0.009 | 0.047 ± 0.007 |
| nmol | | | | |
| Comp. Ex. 1 1 nmol | 0.020 ± 0.006 | 0.035 ± 0.008 | 0.057 ± 0.011 | 0.065 ± 0.010 |
| Comp. Ex. 1 | 0.018 ± 0.004 | 0.042 ± 0.007 | 0.042 ± 0.010 | 0.070 ± 0.007 |
| 3 nmol | | | | |
| Ref. comp. | 0.037 ± 0.005 | 0.063 ± 0.010 | 0.041 ± 0.018 | 0.068 ± 0.022 |
| 3 nmol | | | | |
| Comp. Ex. 2 | 0.025 ± 0.008 | 0.028 ± 0.009 | 0.040 ± 0.009 | 0.061 ± 0.007 |
| 3 nmol | | | | |
| Vehicle | 0.039 ± 0.007 | 0.052 ± 0.008 | 0.067 ± 0.007 | 0.079 ± 0.007 |

### Example 12

### 4—Anti-Inflammatory Properties in a Model of Intravital Microscopy in Mice

The tested compounds are:
(11β,16α)-9-fluoro-11-hydroxy-16,17-[1-methylethylidene bis(oxy)]-21-[1-oxo-[4-(nitrooxymethyl)benzoxy]]pregna-1,4-diene-3,20-dione, prepared as described in example 1;
Triamcinolone acetonide that is the reference compound for the compound of example 1;
(11β)-17-[(ethoxycarbonyl)oxy]-11-hydroxy-21-[1-oxo-[4-(nitrooxymethyl)benzoxy]]pregna-1,4-diene-3,20-dione, prepared as described in example 2;
Prednicarbate that is the reference compound for the compound of example 2;

C57B16 male mice will be anaesthetized by intraperitoneal injection of a mixture of 10 mg/kg xylazine (MTC Pharmaceuticals, Cambridge, Ontario, Canada) and 200 mg/kg ketamine hydrochloride (Rogar/STB, London, Ontario, Canada). Intravital microscopy will be performed on skin flap, which thickness does not allow visualizing leukocyte/endothelial cells interaction by simple trans-illumination. Therefore, after anaesthesia, mice will receive an intravenous injection of a fluorescent dye, rhodamine 6G (Sigma, St. Louis, Mo., USA, 0.3 mg/kg). At this dose, rhodamine 6G labels leukocytes and platelets and has been shown to have no effect on leukocyte kinetics. Then, a midline abdominal incision will be performed, from the diaphragm, extending to the pelvic region. The skin will carefully be separated from the underlying tissue, but remained attached laterally, so the blood supply to the skin flap remained intact. The skin flap will be extended over a viewing pedestal to expose the dermal microvasculature and secured along the edges using 4.0 sutures. The exposed dermal tissues will be superfused with a bicarbonate-buffered saline pH 7.4, to avoid tissue dehydration. The microcirculation will be observed using an inverted microscope (Nikon) with a ×20 objective lens, and rhodamine 6G allows visualization and quantification of the number of rolling and adherent leukocytes, by epi-illumination at 510-560 nm, using a 590-nm emission filter. Single unbranched venules (20-40 μm in diameter) will be selected for the study. Images of the selected venule will be recorded for −5 min, after a 15-min equilibration period and the end of this 5-min interval was considered as time 0. Leukocyte adherence will be determined upon video playback, on 100 μm vessel length (table 4). A leukocyte will be considered adherent to the endothelium if it remained stationary for 30 s or more. Leukocyte flux will be defined as the number of leukocytes per minute moving at a velocity less than that of erythrocytes, which passed a reference point in the venule. The changes in flux of rolling leukocytes will be evaluated as differences between the number of rolling leukocytes at each interval and the basal number of rolling leukocytes (table 5).

The tested compounds were applied topically dissolved in ethanol:sterile water (1:1) and applied at a final volume of 100 μl, five minutes after irritant contact dermatitis will be induced.

Results demonstrated that the compound of the invention displayed statistically significant changes in vessel diameter (Table 4), moreover, both the tested compounds reduced statistically the rolling leukocytes (Table 5), a primary inflammation endpoint, in higher degree than prior-art compounds.

TABLE 4
Changes in vessel diameter

| | Area under the curve |
|---|---|
| Treatment | (change in vessel diameter) |
| Vehicle | 546 ± 135 |
| Compound Ex. 1 | 114 ± 32* |
| (3 nmol) | |
| Triamcinolone | 278 ± 78 |
| (3 nmol) | |
| Compound Ex. 2 | 175 ± 70* |
| (3 nmol) | |
| Prednicarbate | 282 ± 71 |
| (3 nmol) | |

*Statistically different from vehicle

TABLE 5
Rolling leukocytes

| | Area under the curve |
|---|---|
| | (change in flux of rolling |
| Treatment | leucocytes) |
| Vehicle | 941 ± 188 |
| Compound Ex. 1 | 247 ± 35* |
| (3 nmol) | |
| Triamcinolone | 376 ± 129* |
| (3 nmol) | |
| Compound Ex. 2 | 300 ± 75* |
| (3 nmol) | |
| Prednicarbate | 412 ± 112* |
| (3 nmol) | |

*Statistically different from vehicle

What is claimed:

1. ((11β, 16β)-9-fluoro-11-hydroxy-16-methyl-21-[1-oxo-[4(nitrooxymethyl)benzoxyJ-17-(valeryloxy)pregna-1,4-diene-3,20-dione.

2. A method for treating an inflammatory skin condition selected from the group consisting of corticosteroid-responsive dermatosis, inflammation, eczema, erythema, papulation, scaling, erosion, oozing, crusting, pruritis, epidermalysis bullosa, erythema, warts, diaper rash, jock itch, ruber lichen planus, atopic dermatitis, contact dermatitis, psoriasis, and seborrheic dermatitis, comprising the step of applying a therapeutically-effective amount of the compound of claim 1.

3. A method for preparing a pharmaceutical composition for treating atopic dermatitis, comprising the step of providing a therapeutically-effective amount of the compound of claim 1 in a pharmaceutically-acceptable topical formulation.

4. A method for preparing a pharmaceutical composition for treating contact dermatitis, comprising the step of providing a therapeutically-effective amount of the compound of claim 1 in a pharmaceutically-acceptable topical formulation.

5. A method for preparing a pharmaceutical composition for treating psoriasis, comprising the step of providing a therapeutically-effective amount of the compound of claim 1 in a pharmaceutically-acceptable topical formulation.

6. A topical pharmaceutical formulation comprising the compound of claim 1 and one or more pharmaceutically-acceptable excipients.

7. The topical pharmaceutical formulation of claim 6, wherein the pharmaceutical formulation is provided in a form selected from the group consisting of creams, lotions, ointments, and sprays.
